(19) United States
(12) Patent Application Publication
(10) Pub. No.: US 2024/0043612 A1
(43) Pub. Date: Feb. 8, 2024

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventor: Ayano ASANO, Tokyo (JP)

(73) Assignee: Resonac Corporation, Tokyo (JP)

(21) Appl. No.: 18/038,755

(22) PCT Filed: Nov. 17, 2021
(86) PCT No.: PCT/JP2021/042288

§ 371 Date: May 25, 2023

(30) Foreign Application Priority Data
Nov. 27, 2020 (JP) 2020-197545

(51) Int. Cl. C08G 65/22; C10M 107/38; G11B 5/725

(52) U.S. Cl. CPC C08G 65/226; C10M 107/38; G11B 5/7257; C08G 2650/38; C08G 2650/48; C10M 2213/0606; C10N 2020/04

(57) ABSTRACT

A fluorine-containing ether compound represented by the following formula is provided. R¹—CH₂—R²—CH₂—R³—CH₂—R⁴—CH₂—R⁵

(In the formula, R³ is a divalent organic group containing at least one polar group and an alicyclic structure having 3 to 13 carbons, and does not contain a perfluoropolyether chain, R² and R⁴ are perfluoropolyether chains, and R¹ and R⁵ are terminal groups containing two or three polar groups, in which individual polar groups are bound to different carbon atoms and the carbon atoms to which the polar groups are bound are bound to each other via a linking group containing a carbon atom to which the polar groups are not bound.)

The present invention relates to a fluorine-containing ether compound, a lubricant for a magnetic recording medium, and a magnetic recording medium.

Priority is claimed on Japanese Patent Application No. 2020-197545, filed Nov. 27, 2020, the content of which is incorporated herein by reference.

### BACKGROUND ART

Development of magnetic recording media suitable for high recording densities is underway to improve the recording densities of magnetic recording/reproducing devices.

As a conventional magnetic recording medium, there is a magnetic recording medium in which a recording layer is formed on a substrate and a protective layer made of carbon or the like is formed on the recording layer. The protective layer protects information recorded in the recording layer and enhances the slidability of a magnetic head. In addition, the protective layer covers the recording layer to prevent metal contained in the recording layer from being corroded by environmental substances.

However, sufficient durability of the magnetic recording medium cannot be obtained by simply providing the protective layer on the recording layer. Therefore, a lubricant is applied to the surface of the protective layer to form a lubricating layer with a thickness of about 0.5 to 3 nm. The lubricating layer improves the durability and protective power of the protective layer and prevents contamination substances from intruding into the magnetic recording medium.

As a lubricant used at the time of forming a lubricating layer in a magnetic recording medium, there is a fluorine-based polymer having a repeating structure containing —CF₂—. As a fluorine-based polymer, a polymer wherein a compound having a polar group such as a hydroxyl group at a terminal is linked with a saturated alicyclic structure has been proposed.

For example, a fluorine-containing ether compound in which three fluorine-containing ether groups each having polar groups at a terminal are connected to a trivalent atom or a trivalent atom group is disclosed in Patent Document 1.

A fluorine-containing ether compound having an alicyclic hydrocarbon group near a center portion and polar groups at a terminal is disclosed in Patent Document 2.

### CITATION LIST

### Patent Literature

Patent Document 1: PCT International Publication No. WO2018/159232
Patent Document 2: PCT International Publication No. WO2013/054393

### SUMMARY OF INVENTION

### Technical Problem

There is a demand for a further decrease in the flying height of a magnetic head in magnetic recording/reproducing devices. This requires a further decrease in the thickness of lubricating layers in magnetic recording media.

However, when the thickness of lubricating layers is reduced, the coatability of the lubricating layers tends to decrease, and the resistance to chemical substances of magnetic recording media and the wear resistance of the lubricating layers tend to decrease.

The present invention has been made in consideration of the above circumstances, and an object of the invention is to provide a suitable fluorine-containing ether compound as a material for a lubricant for a magnetic recording medium with which a lubricating layer having excellent resistance to chemical substances and wear resistance can be formed even if the lubricating layer is thin.

In addition, another object of the present invention is to provide a lubricant for a magnetic recording medium containing the fluorine-containing ether compound of the present invention.

In addition, still another object of the present invention is to provide a magnetic recording medium which has a lubricating layer containing the fluorine-containing ether compound of the present invention and has excellent reliability and durability.

### Solution to Problem

The present inventors have conducted extensive studies in order to solve the above-described problem.

As a result, they have found that a fluorine-containing ether compound may suffice in which an organic group containing an alicyclic structure having 3 to 13 carbons and at least one polar group is placed in the center of a molecule, and perfluoropolyether chains, methylene groups, and terminal groups having a specific structure having two or three polar groups are sequentially bound to both sides of the organic group via methylene groups, thus leading to realization of the present invention.

That is, the present invention relates to the following features.

A first aspect of the present invention provides the following fluorine-containing ether compound.

[1] A fluorine-containing ether compound represented by Formula (1) below.

R¹—CH₂-R²—CH₂-R³—CH₂-R⁴—CH₂-R⁵ (1)
(In Formula (1). R³ is a divalent organic group containing at least one polar group and an alicyclic structure having 3 to 13 carbons, and does not contain a perfluoropolyether chain, R² and R⁴ are perfluoropolyether chains, and R¹ and R⁵ are terminal groups containing two or three polar groups, in which individual polar groups are bound to different carbon atoms and the carbon atoms to which the polar groups are bound are bound to each other via a linking group containing a carbon atom to which the polar groups are not bound.)

The compound of the first aspect of the present invention preferably includes features described in [2] to [11] below. It is also preferable to arbitrarily combine two or more of the features described in [2] to [11] below.

[2] The fluorine-containing ether compound according to [1], in which R³ above is represented by any of Formulae (2-1) to (2-4) below.

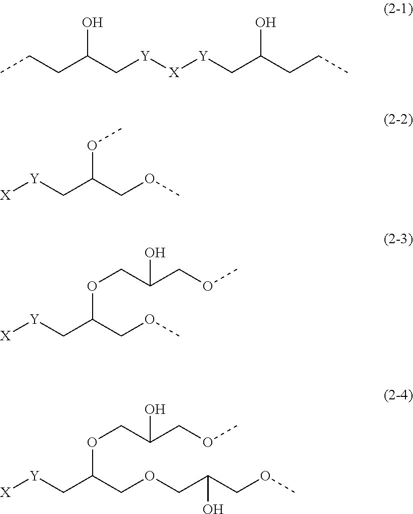

(In Formula (2-1), X is an alicyclic structure having 3 to 13 carbons, and Y represents —O—, —NH—, or —CH₂—.)

(In Formula (2-2), X′ is an alicyclic structure having 3 to 13 carbons and has at least one substituent containing a polar group, and Y represents —O—. —NH—, or ˜CH₂—.)

(In Formula (2-3), X is an alicyclic structure having 3 to 13 carbons, and Y represents —O—, —NH—, or —CH₂—.)

(In Formula (2-4), X is an alicyclic structure having 3 to 13 carbons, and Y represents —O—, —NH—, or —CH₂—.)

The fluorine-containing ether compound according to [2], in which Y in Formulae (2-1) to (2-4) is —O—.

[4] The fluorine-containing ether compound according to any one of [1] to [3], in which the alicyclic structure contained in R³ above is a saturated alicyclic structure.

[5] The fluorine-containing ether compound according to any one of [1] to [4], in which the alicyclic structure contained in R³ above is selected from a group consisting of cyclopropane, cyclobutane, cyclopentane, cyclohexane, cycloheptane, cyclooctane, cyclononane, cyclodecane, cycloundecane, cyclododecane, cyclotridecane, and adamantane.

[6] The fluorine-containing ether compound according to any one of [1] to [5], in which the at least one polar group contained in R³ above is a group containing a polar group selected from a group consisting of a hydroxyl group, an alkoxy group, an amide group, an amino group, a carbonyl group, a carboxy group, a nitro group, a cyano group, and a sulfo group.

[7] The fluorine-containing ether compound according to any one of [1] to [6], in which R² and R⁴ above are any of Formulae (4) to (6) below.

—CF₂O—(CF₂CF₂O)_{b}—(CF₂O)_{c}—CF₂— (4)
(b and c in Formula (4) indicate an average degree of polymerization, each independently representing 0 to 30, provided that b and c are not 0 at the same time.)

—CF(CF₃)(OCF(CF₃)CF₂)_{d}—OCF(CF₃) (5)
(d in Formula (5) indicates an average degree of polymerization and represents 0.1 to 30.)

—CF₂CF₂O—(CF₂CF₂CF₂O)ₑ—CF₂CF₂— (6)
(e in Formula (6) indicates an average degree of polymerization and represents 0.1 to 30.)

[8] The fluorine-containing ether compound according to any one of [1] to [7], in which the two or three polar groups contained in R¹ and R⁵ above are all hydroxyl groups.

[9] The fluorine-containing ether compound according to any one of [1] to [8], in which R¹ and R⁵ above are terminal groups represented by any of Formulae (7) to (10) below.

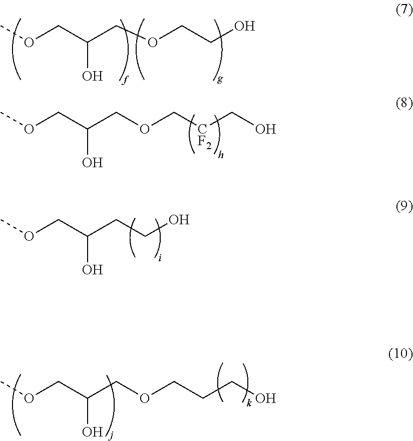

(In Formula (7), f represents an integer of 1 to 2, and g represents an integer of 1 to 5.)

(In Formula (8), h represents an integer of 1 to 5.)

(In Formula (9), i represents an integer of 1 to 5.)

(In Formula (0), j represents an integer of 1 to 2, and k represents an integer of 1 to 2.)

[10] The fluorine-containing ether compound according to any one of [1] to [9], in which a number-average molecular weight thereof is within a range of 500 to 10,000.

[11] The fluorine-containing ether compound according to any one of [1] to [10)], in which the compound represented by Formula (1) above is any of compounds represented by Formulae (A) to (P) below.

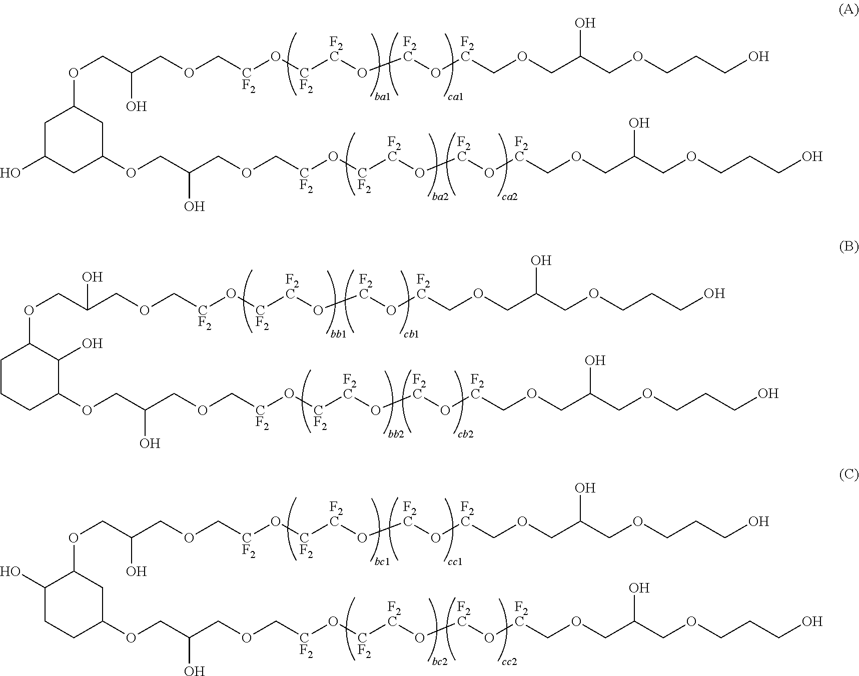

(In Formula (A), ba1, ca1, ba2, and ca2 indicate an average degree of polymerization, ba1 and ba1 represent 0 to 30, and ca1 and ca2 represent 0 to 30, provided that ba1 and ca1 are not 0 at the same time and ba2 and ca2 are not 0 at the same time.)

(In Formula (B), bb1, cb1, bb2, and cb2 indicate an average degree of polymerization, bb1 and bb2 represent 0 to 30, and cb1 and cb2 represent 0 to 30, provided that bb1 and cb1 are not 0 at the same time and bb2 and cb2 are not 0 at the same time.)

(In Formula (C), bc1, cc1, bc2, and cc2 indicate an average degree of polymerization, bc1 and bc2 represent 0 to 30, and cc1 and cc2 represent 0 to 30, provided that bc1 and cc1 are not 0 at the same time and bc2 and cc2 are not 0 at the same time.)

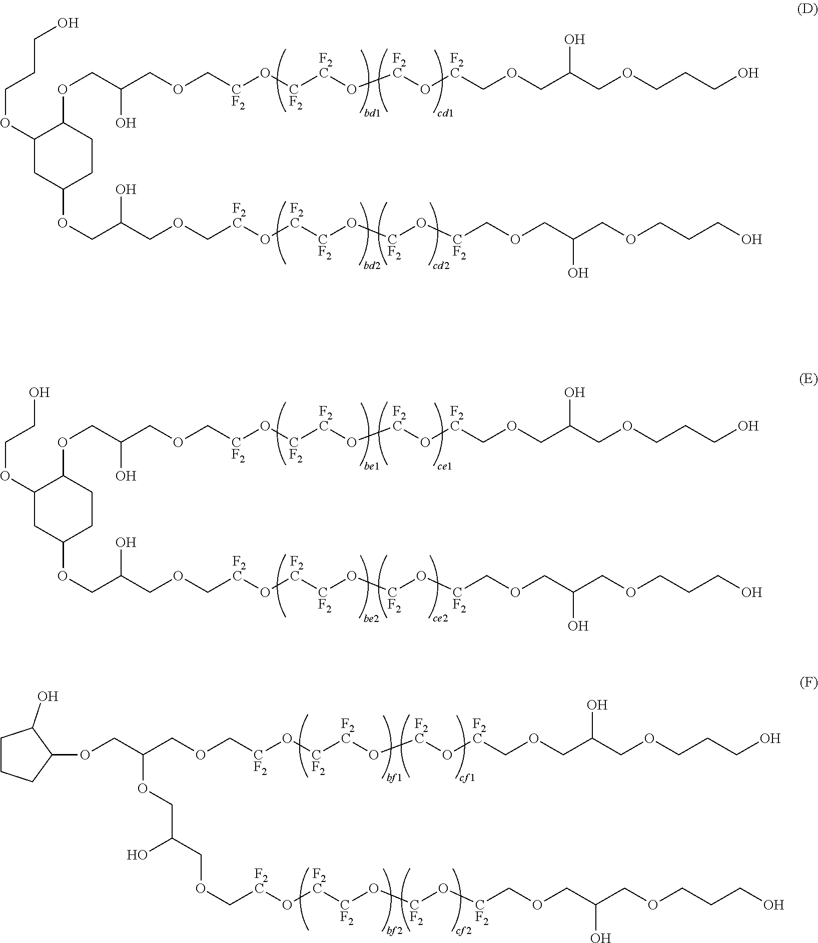

(In Formula (D), bd1, cd1, bd2, and cd2 indicate an average degree of polymerization, bd1 and bd2 represent 0 to 30, and cd1 and cd2 represent 0 to 30, provided that bd1 and cd1 are not 0 at the same time and bd2 and cd2 are not 0 at the same time.)

(In Formula (E), be1, ce1, be2, and ce2 indicate an average degree of polymerization, be1 and be2 represent 0 to 30, and ce1 and ce2 represent 0 to 30, provided that be1 and ce1 are not 0 at the same time and bf2 and cf2 are not 0 at the same time.)

(In Formula (F), bf1, cf1, bf2, and cf2 indicate an average degree of polymerization, bf1 and bf2 represent 0 to 30, and cf1 and cf2 represent 0 to 30, provided that bf1 and cf1 are not 0 at the same time and bf2 and cf2 are not 0 at the same time.)

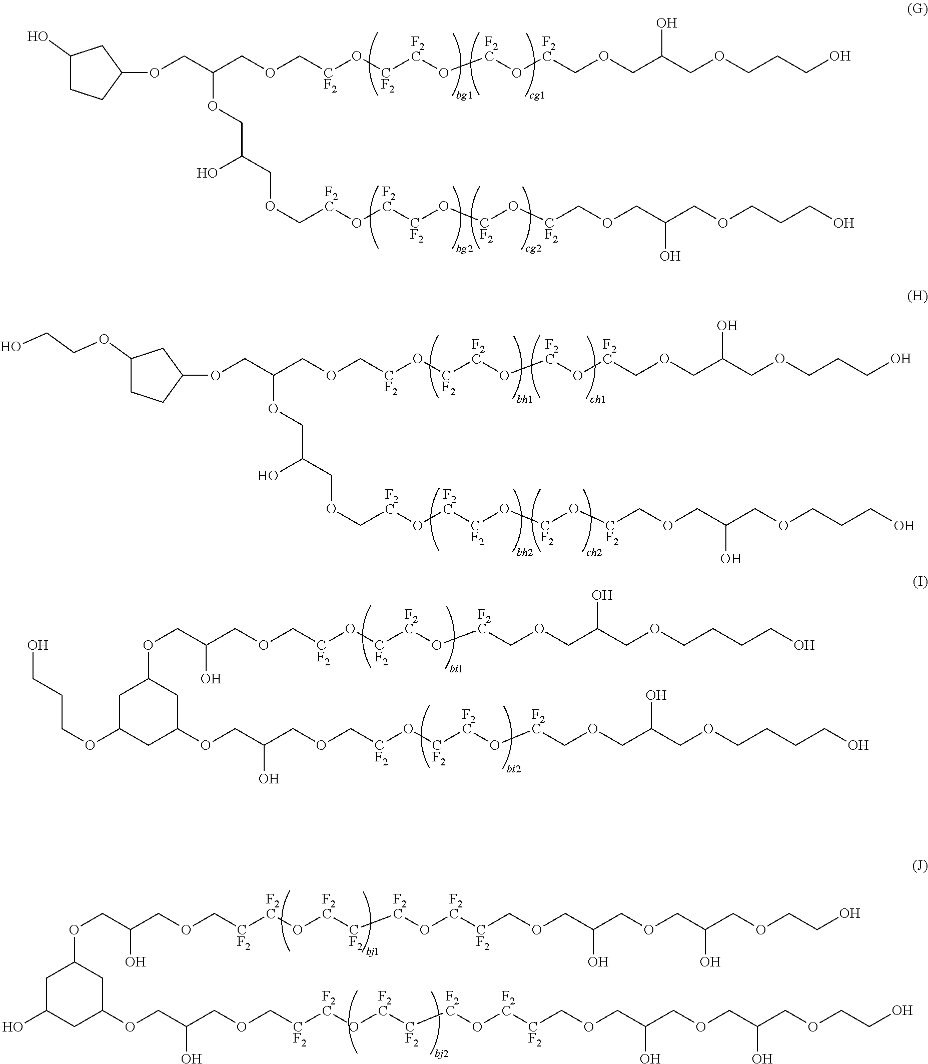

(In Formula (G), bg1, cg1, bg2, and cg2 indicate an average degree of polymerization, bg1 and bg2 represent 0 to 30, and cg1 and cg2 represent 0 to 30, provided that bg1 and cg1 are not 0 at the same time and bg2 and cg2 are not 0 at the same time.)

(In Formula (H), bh1, ch1, bh2, and ch2 indicate an average degree of polymerization, bh1 and bh2 represent 0 to 30, and ch1 and ch2 represent 0 to 30, provided that bh1 and ch1 are not 0 at the same time and bh2 and ch2 are not 0 at the same time.)

(In Formula (I), bi1 and bi2 indicate an average degree of polymerization and represent 0.1 to 30.)

(In Formula (J), ej1 and ej2 indicate an average degree of polymerization and represent 0.1 to 30.)

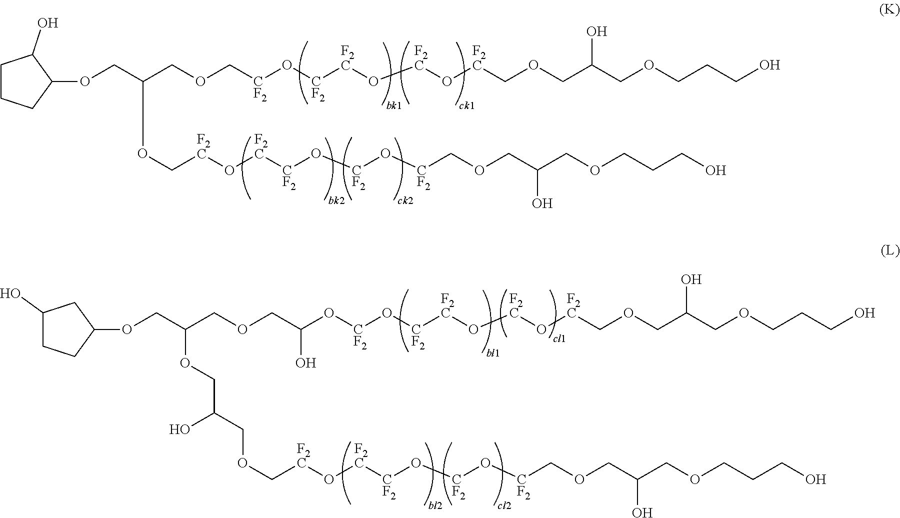

(In Formula (K), bk1, ck1, bk2, and ck2 indicate an average degree of polymerization, bk1 and bk2 represent 0 to 30, and ck1 and ck2 represent 0 to 30, provided that bk1 and ck1 are not 0 at the same time and bk2 and ck2 are not 0 at the same time.)

(In Formula (L), bl1, cl1, bl2, and cl2 indicate an average degree of polymerization, bl1 and bl2 represent 0 to 30, and cl1 and cl2 represent 0 to 30, provided that bl1 and cl1 are not 0 at the same time and bl2 and cl2 are not 0 at the same time.)

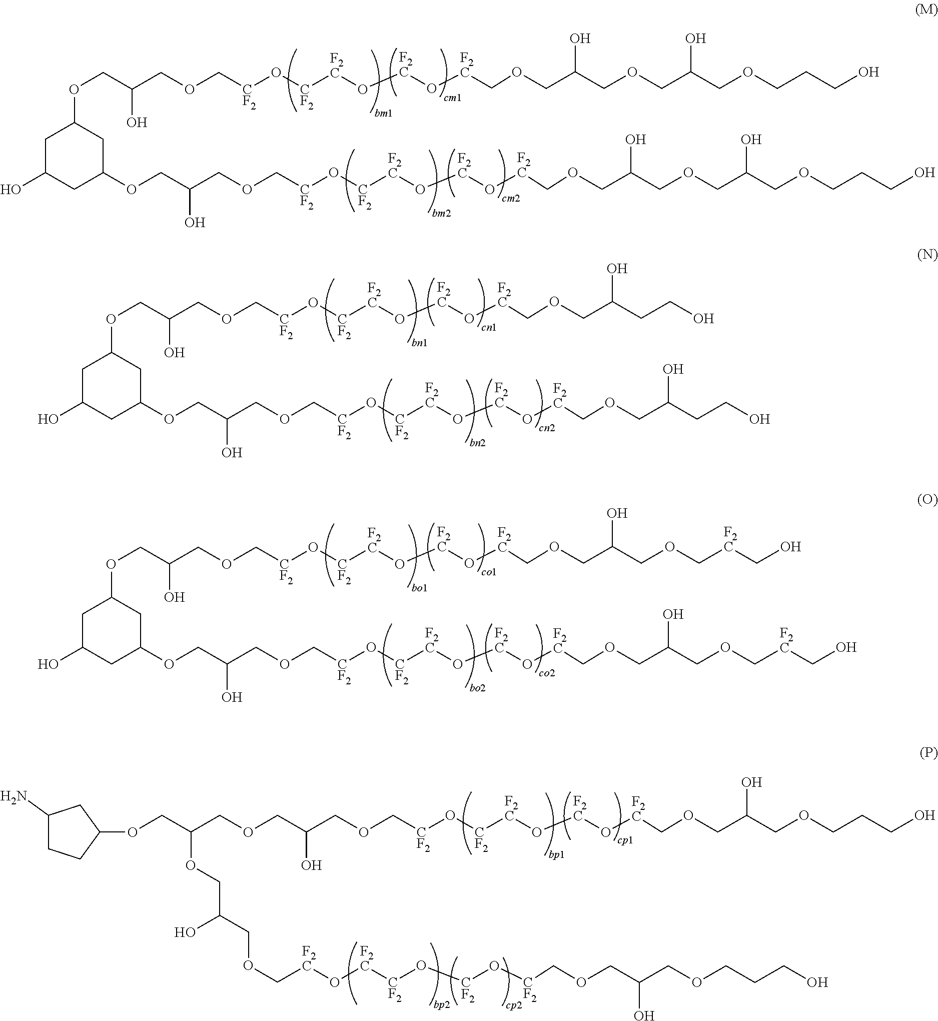

(In Formula (M), bm1, cm1, bm2, and cm2 indicate an average degree of polymerization, bm1 and bm2 represent 0 to 30, and cm1 and cm2 represent 0 to 30, provided that bm1 and cm1 are not 0 at the same time and bm2 and cm2 are not 0 at the same time.)

(In Formula (N), bn1, cn1, bn2, and cn2 indicate an average degree of polymerization, bn1 and bn2 represent 0 to 30, and cn1 and cn2 represent 0 to 30, provided that bn1 and cn1 are not 0 at the same time and bn2 and cn2 are not 0 at the same time.)

(In Formula (O), bo1, co1, bo2, and co2 indicate an average degree of polymerization, bo1 and bo2 represent 0 to 30, and co1 and co2 represent 0 to 30, provided that bo1 and co1 are not 0 at the same time and bo2 and co2 are not 0 at the same time.)

(In Formula (P), bp1, cp1, bp2, and cp2 indicate an average degree of polymerization, bp1 and bp2 represent 0 to 30, and cp1 and cp2 represent 0 to 30, provided that bp1 and cp1 are not 0 at the same time and bp2 and cp2 are not 0 at the same time.)

A second aspect of the present invention is to provide a lubricant for a magnetic recording medium below.

[12] A lubricant for a magnetic recording medium, including: the fluorine-containing ether compound according to any one of [1] to [11].

A third aspect of the present, invention is to provide a magnetic recording medium below.

[13] A magnetic recording medium, in which at least a magnetic layer, a protective layer, and a lubricating layer are sequentially provided on a substrate, and the lubricating layer contains the fluorine-containing ether compound according to any one of [1] to [11].

The third aspect of the present invention preferably has the following feature.

[14] The magnetic recording medium according to [13], in which an average film thickness of the lubricating layer is 0.5 nm to 2.0 nm.

### Advantageous Effects of Invention

A fluorine-containing ether compound of the present invention is a compound represented by Formula (1) above and is suitable as a material for a lubricant for a magnetic recording medium.

Since the lubricant for a magnetic recording medium of the present invention contains the fluorine-containing ether compound of the present invention, it is possible to form a lubricating layer having excellent resistance to chemical substances and wear resistance even if the lubricating layer is thin.

A magnetic recording medium of the present invention has a lubricating layer with excellent resistance to chemical substances and wear resistance, and thus has excellent reliability and durability.

### BRIEF DESCRIPTION OF DRAWINGS

FIG. 1 is a schematic cross-sectional view showing an example of one preferred embodiment of a magnetic recording medium of the present invention.

### DESCRIPTION OF EMBODIMENT

Hereinafter, a fluorine-containing ether compound, a lubricant for a magnetic recording medium (hereinafter, abbreviated as a “lubricant” in some cases), and a magnetic recording medium of the present invention will be described in detail. The present invention is not limited to only the embodiment shown below. For example, the present invention is not limited to only the following examples. Within the scope not departing from the gist of the present invention, numbers, quantities, ratios, compositions, types, positions, materials, configurations, and the like can be added, omitted, substituted, or changed.

[Fluorine-Containing Ether Compound]

A fluorine-containing ether compound of the present embodiment is represented by Formula (1) below.

R¹—CH₂-R²—CH₂-R³—CH₂-R⁴—CH₂—R⁵ (1)

(In Formula (1), R³ is a divalent organic group containing at least one polar group and an alicyclic structure having 3 to 13 carbons, and does not contain a perfluoropolyether chain, R² and R⁴ are perfluoropolyether chains, and R¹ and R⁵ are terminal groups containing two or three polar groups, in which individual polar groups are bound to different carbon atoms and the carbon atoms to which the polar groups are bound are bound to each other via a linking group containing a carbon atom to which no polar group is bound.)

Here, the reason why it is possible to form a lubricating layer with excellent resistance to chemical substances and wear resistance even if the lubricating layer is thin in a case where the lubricating layer is formed on a protective layer of a magnetic recording medium using a lubricant containing the fluorine-containing ether compound of the present embodiment will be described.

The fluorine-containing ether compound of the present embodiment has perfluoropolyether chains (hereinafter sometimes abbreviated as “PFPE chains”) represented by R² and R³ as shown in Formula (1). Due to PFPE chains, in a case where a lubricant containing a fluorine-containing ether compound is applied onto a protective layer to form a lubricating layer, the surface of the protective layer is covered and lubricity is imparted to the lubricating layer to reduce frictional force between a magnetic head and the protective layer.

In addition, as shown in Formula (1), a divalent organic group which contains an alicyclic structure having 3 to 13 carbons and at least one polar group and is represented by R is placed at end portions (first end portions) of the PFPE chains represented by R² and R⁴ via methylene groups (—CH₂—). The alicyclic structure having 3 to 13 carbons contained in R³ is moderately bulky, and therefore imparts moderate fluidity to the molecular structure of the fluorine-containing ether compound represented by Formula (1). As a result, in the lubricating layer containing the fluorine-containing ether compound of the present embodiment, a part of the alicyclic structure contained in R³ can be lifted from the protective layer. As a result, before the magnetic head collides with the protective layer, the lubricating layer collides with the magnetic head to protect the protective layer. Due to such a function, the alicyclic structure contained in R³ improves the wear resistance of the lubricating layer containing the fluorine-containing ether compound of the present embodiment.

In addition, R³ shown in Formula (1) is a divalent organic group containing at least one polar group. The polar group contained in R³ has a pinning effect that prevents the bulky alicyclic structure having 3 to 13 carbons from being lifted excessively from the protective layer. Accordingly, the polar group contained in R³ contributes to adhesion properties with respect to the protective layer, on which a lubricant containing the fluorine-containing ether compound of the present embodiment is applied, and the lubricating layer formed through application of the lubricant.

In addition, as shown in Formula (1), terminal groups containing two or three polar groups and represented by R¹ and R⁵ are placed at end portions (second end portions) of the ITP chains represented by R² and R⁴ on opposite sides to 1W via methylene groups (—CH₂—). The terminal groups represented by R¹ and R⁵ contribute to adhesion properties with respect to the protective layer, on which a lubricant containing the fluorine-containing ether compound of the present embodiment is applied, and the lubricating layer formed through application of the lubricant. The two or three polar groups contained in the terminal groups represented by R¹ and R⁵ exhibit excellent resistance to chemical substances in the lubricating layer containing the fluorine-containing ether compound, by making the protective layer and the fluorine-containing ether compound of the present embodiment adhere closely to each other.

In addition, the two or three polar groups contained in the terminal groups represented by R¹ and R⁵ are bound to different carbon atoms, and the carbon atoms to which the polar groups are bound are bound to each other via a linking group containing a carbon atom to which the polar groups are not bound. For this reason, the two or three polar groups contained in R¹ and R⁵ have appropriate distances therebetween. As a result, the fluorine-containing ether compound having the terminal groups represented by R¹ and R⁵ is less likely to aggregate compared to for example, a fluorine-containing ether compound in which at least some carbon atoms to which polar groups contained in terminal groups represented by R¹ and R⁵ are directly bound to each other. Moreover, in the fluorine-containing ether compound represented by Formula (1), R³ does not contain a perfluoropolyether chain. For this reason, aggregation is less likely to occur compared to a case where, for example, R³ contains a perfluoropolyether chain.

Fluorine-containing ether compounds of the present embodiment are less likely to aggregate in this manner, and therefore are likely to be placed on the protective layer in a state in which they spread in the surface direction and extend uniformly. For this reason, the lubricant containing the fluorine-containing ether compound of the present embodiment can cover the surface of the protective layer with a high coating rate and can form the lubricating layer having excellent resistance to chemical substances even if the lubricating layer is thin. Accordingly, the lubricant containing the fluorine-containing ether compound of the present embodiment contributes to thinning of the lubricating layer (reduction in magnetic spacing).

From the above, it is inferred that the lubricant containing the fluorine-containing ether compound of the present embodiment can cover the surface of the protective layer with a high coating rate and can form the lubricating layer having excellent resistance to chemical substances and wear resistance even if the lubricating layer is thin.

(Organic Group Represented by R³)

In the fluorine-containing ether compound of the present embodiment represented by Formula (1), the organic group represented by R³ has an alicyclic structure having 3 to 13 carbons. The number of carbon atoms can be arbitrarily selected within this range, and may be, for example, 3 to 6, 7 to 9, or 10 to 13. The alicyclic structure having 3 to 13 carbons is preferably a saturated alicyclic structure to obtain a fluorine-containing ether compound with which a lubricating layer having superior wear resistance is obtained. The saturated alicyclic structure may be a bridged saturated alicyclic structure. When R³ has a saturated alicyclic structure, the molecular structure of the fluorine-containing ether compound represented by Formula (1) has superior fluidity. Accordingly, the saturated alicyclic structure portion in the lubricating layer containing the fluorine-containing ether compound is more likely to be lifted from the protective layer, and the protective layer can be effectively protected by the lifted lubricating layer.

Specific preferable examples of saturated alicyclic structures having 3 to 13 carbons include any one selected from cyclopropane, cyclobutane, cyclopentane, cyclohexane, cycloheptane, cyclooctane, cyclononane, cyclodecane, cycloundecane, cyclododecane, cyclotridecane, and adamantane. In the alicyclic structure having 3 to 13 carbons in the organic group represented by R³, a saturated alicyclic structure having 4 to 8 carbons is particularly preferable to obtain a fluorine-containing ether compound with which a lubricating layer having superior wear resistance is obtained.

The number of alicyclic structures having 3 to 13 carbons in the organic group represented by R³ may be only one or a plurality. For example, in a case where the number of alicyclic structures is a plurality, it may be 2 to 6, 3 to 5, and the like, and is not limited to these examples. In the case where the number of alicyclic structures having 3 to 13 carbons in the organic group represented by R is a plurality, some or all of the plurality thereof may be the same as or different from each other. The number of alicyclic structures having 3 to 13 carbons in the organic group represented by R³ is preferably small and most preferably only 1 because in this case the fluorine-containing ether compounds are less likely to aggregate.

An alicyclic structure having 3 to 13 carbons in the organic group represented by R³ may have one or more substituents. In a case where the alicyclic structure has two or more substituents, some or all of the two or more substituents may be the same as or different from each other. In a case where the alicyclic structure having 3 to 13 carbons has substituents, the number of substituents is not particularly limited and can be appropriately determined depending on the type of alicyclic structure having 3 to 13 carbons. The number of carbon atoms in a substituent is not included in the number of carbon atoms in the alicyclic structure.

A substituent in the case where the alicyclic structure having 3 to 13 carbons has the substituent is preferably a substituent having 0 to 10 carbons. When the number of carbon atoms in the substituent is 0 to 10, the substituent of the alicyclic structure does not become a steric hindrance due to an excessive number of carbon atoms in the substituent. Accordingly, a lubricating layer with favorable coatability can be obtained without suppressing adsorption power of the lubricating layer with respect to a protective layer due to the substituent of the alicyclic structure. The number of carbon atoms in the substituent may be 1 to 8 or 2 to 6. The number of carbon atoms in the substituent is more preferably 0 to 5 and still more preferably 0 to 3.

A substituent in the case where the alicyclic structure having 3 to 13 carbons has the substituent is preferably a substituent containing a polar group. Specific examples thereof include: functional groups selected from the group consisting of a hydroxyl group, an alkoxy group, an amide group, an amino group, a carbonyl group, a carboxy group, a nitro group, a cyano group, and a sulfo group: and an alkyl group having one or more selected from the functional groups. With respect to the alkyl group having the functional groups, the number of carbon atoms in the alkyl group is preferably 1 to 3 and more preferably 2 or 3.

Among these substituents, substituents selected from a hydroxyl group, an amide group, an amino group, a cyano group, and an alkoxy group or an alkyl group having any one selected from these functional groups are more preferable. Specific examples of the substituents include —OH, —CH₂OH, —CH₂CH₂OH, and —CH₂CH₂CH₂OH; —OCH₃, —OCH₂CH₃, and —OCH₂CH₂CH₃; —OCH₂OH, —OCH₂CH₂OH, and —OCH₂CH₂CH₂OH; —CONH₂, CH₂CONH₂, and —CH₂CH₂CONH₂; —NH₂, —CH₂NH₂, —CH₂CH₂NH₂, and —CH₂CH₂CH₂NH₂; and —CN, —CH₂CN, and —CH₂CH₂CN.

Among these substituents, substituents selected from a hydroxyl group, an amino group, an amide group, and an alkoxy group or an alkyl group having any one selected from these polar groups are particularly preferable because they are polar groups capable of hydrogen bonding. In a case where the alicyclic structure having 3 to 13 carbons has one or more substituents selected therefrom, the adsorption power of the lubricating layer with respect to the protective layer further increases due to interaction between the above-described substituents and the protective layer which is placed in contact with the lubricating layer containing the fluorine-containing ether compound. As a result, the lubricant containing the fluorine-containing ether compound has superior resistance to chemical substances and wear resistance, which is preferable.

In particular, a substituent in the case where the alicyclic structure having 3 to 13 carbons has the substituent is preferably a substituent wherein a carbon atom to which a polar group in the substituent is bound is bound to the alicyclic structure via a linking group containing an ether bond and a carbon atom. Examples of such a substituent include an alkoxy group having polar groups at a terminal, and specific examples thereof include —OCH₂CH₂OH and —OCH₂CH₂CH₂OH. In the ease where a carbon atom to which a polar group in the substituent is bound is bound to the alicyclic structure via a linking group containing an ether bond and a carbon atom, the distance between the alicyclic structure and the polar group in the substituent is sufficiently ensured in R by the linking group having moderate flexibility. As a result, the pinning effect of the alicyclic structure due to the polar group in the substituent is appropriate, and a fluorine-containing ether compound with which a lubricating layer having superior wear resistance can be formed is obtained.

In the fluorine-containing ether compound represented by Formula (1), the organic group represented by R³ contains at least one polar group. The polar group contained in R³ may be bound to a linking group which binds the alicyclic structure to —CH₂— (methylene groups) bound to perfluoropolyether chains represented by R² and R⁴, or may be a substituent of the alicyclic structure. R³ preferably has both a polar group as the substituent of the alicyclic structure and a polar group bound to a linking group which binds the alicyclic structure to —CH₂— bound to R² and R⁴.

At least one polar group contained in R³ is preferably a group containing a polar group selected from the group consisting of a hydroxyl group, an alkoxy group, an amide group, an amino group, a carbonyl group, a carboxy group, a nitro group, a cyano group, and a sulfo group. An ether bond (—O—) is not considered as a polar group in R³. Among the above, in particular, at least one polar group contained in R is preferably a group containing a hydroxyl group or an amino group and more preferably a group containing a hydroxyl group.

The number of the polar group contained in R³ is preferably 1 to 3 and more preferably 2 or 3. If the number of the polar group is 3 or less, the fluidity of the entire molecule due to the inclusion of an alicyclic structure in R³ is not excessively weakened by too strong a pinning effect due to the polar group contained in R³. That is, the pinning effect does not become too strong. In a case where R³ contains two or more polar groups, the types of polar groups may be different from each other or all may be the same as each other, and all are preferably hydroxyl groups.

The organic group represented by R³ in Formula (1) does not contain a perfluoropolyether chain. For this reason, the fluorine-containing ether compounds represented by Formula (1) are less likely to aggregate compared to fluorine-containing ether compounds in which R³ contains a perfluoropolyether chain. As a result the fluorine-containing ether compounds represented by Formula (1) are likely to be placed on a protective layer in a state in which they spread in the surface direction and extend uniformly, and have excellent resistance to chemical substances and wear resistance compared to fluorine-containing ether compounds in which R contains a perfluoropolyether chain, which is preferable.

In the fluorine-containing ether compound represented by Formula (1), the organic group represented by R is bound to perfluoropolyether chains represented by R² and R⁴ via —CH₂— (methylene groups). From the viewpoint of ease of synthesis, the methylene groups bound to R² and R⁴ are preferably bound to any of nitrogen atoms, oxygen atoms, and carbon atoms contained in R and more preferably bound to nitrogen atoms or oxygen atoms contained in R³. In particular, the methylene groups bound to R² and R⁴ are preferably bound to oxygen atoms contained in R³. In this case, the molecular structure of the fluorine-containing ether compound represented by Formula (1) has moderate flexibility. As a result, the lubricating layer containing the fluorine-containing ether compound represented by Formula (1) has superior adhesion properties with respect to a protective layer.

R³ shown in Formula (1) is preferably represented by any of Formulae (2-1) to (2-4) below. In this case, the lubricating layer containing the fluorine-containing ether compound has superior resistance to chemical substances and wear resistance.

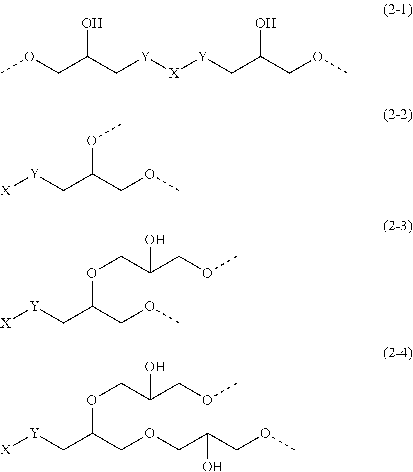

(In Formula (2-1), X is an alicyclic structure having 3 to 13 carbons, and Y represents —O—, —NH—, or —CH₂—.)

(In Formula (2-2). X′ is an alicyclic structure having 3 to 13 carbons and has at least one substituent containing a polar group, and Y represents —O—, —NH—, or —CH₂—.)

(In Formula (2-3), X is an alicyclic structure having 3 to 13 carbons, and Y represents —O—, —NH—, or —CH₂—.)

(In Formula (2-4), X is an alicyclic structure having 3 to 13 carbons, and Y represents —O—, —NH—, or —CH₂—.)

In Formulae (2-1) to (2-4) above, Y represents —O—, —NH—, or —CH₂—. Y is preferably —O— (ether bond) because of easy procurement of raw materials of fluorine-containing ether compounds and appropriate fluidity of molecular structures of fluorine-containing ether compounds.

In Formulae (2-1) to (2-4) above, X and X′ contain the above-described alicyclic structure having 3 to 13 carbons. That is, X and X′ in these formulae may have the above-described alicyclic structure having 3 to 13 carbons and can have the characteristics thereof.

The binding sites of Y in the alicyclic structures X and X′ in Formulae (2-1) to (2-4) are not particularly limited, and Y may be bound to any carbon atom constituting the alicyclic structures X and X′.

In a case where R is any of Formulae (2-1), (2-3), and (2-4), the alicyclic structure X in R³ is bound to carbon atoms to which hydroxyl groups in R³ are bound, via a linking group containing Y and a carbon atom. As a result, the distance between the hydroxyl group and the alicyclic structure X in R³ is sufficiently ensured by the linking group having moderate flexibility. As a result, the pinning effect of the alicyclic structure X due to the hydroxyl group in R; is sufficiently obtained, and a fluorine-containing ether compound with which a lubricating layer having superior wear resistance can be formed is obtained.

In a case where R³ is Formula (2-2). X′ is the above-described alicyclic structure having 3 to 13 carbons and has at least one substituent containing a polar group. The alicyclic structure X′ when R³ is Formula (2-2) contains at least one substituent containing a polar group, and may further have a substituent containing no polar group. In the case where Rx is Formula (2-2), the number of substituent containing a polar group in the alicyclic structure X′ is the number of polar group contained in R³ described above. In the case where R³ is Formula (2-2), the number of the substituent containing a polar group in the alicyclic structure X′ is at least one, preferably 1 to 3, and more preferably 1 or 2.

The substituent containing a polar group in the alicyclic structure X′ in Formula (2-2) is preferably a substituent containing a hydroxyl group, and specifically, any one selected from —OH, —CH₂OH, —CH₂CH₂OH, —CH₂CH₂CH₂OH, —OCH₂OH, —OCH₂CH₂OH, and —OCH₂CH₂CH₂OH is preferable. When the substituent containing a polar group in the alicyclic structure X′ in Formula (2-2) are any substituents selected therefrom, the pinning effect of preventing the bulky alicyclic structure X′ contained in the lubricating layer containing the fluorine-containing ether compound from completely being lifted from the protective layer is more effectively obtained.

(Perfluoropolyether Chains Represented by R² and R⁴)

R² and R⁴ in the fluorine-containing ether compound represented by Formula (1) are perfluoropolyether chains (PFPE chains). The fluorine-containing ether compound represented by Formula (1) preferably contains only two PFPE chains R and R⁴ in the molecule. That is. R¹ and R⁵ in Formula (1) preferably do not contain a PFPE chain. When the number of PFPE chains contained in the molecule is only two, the fluorine-containing ether compounds are less likely to aggregate. For this mason, the lubricating layer containing the fluorine-containing ether compounds represented by Formula (1) is likely to be placed on the protective layer in a state in which the fluorine-containing ether compounds spread in the surface direction and extend uniformly, which is preferable.

The PFPE chains represented by R⁷ and R⁴ are not particularly limited and can be appropriately selected depending on the performance and the like required of a lubricant containing a fluorine-containing ether compound.

R² and R⁴ may be the same as or different from each other. It is preferable that R² and R⁴ be the same perfluoropolyether chains because a fluorine-containing ether compound is easily synthesized.

The PFPE chains may have a structure represented by Formula (Rf) below derived front a perfluoroalkylene oxide polymer or copolymer, for example.

—(CF₂)_{w1}O(CF₂O)_{w2}(CF₂CF₂O)_{w3}(CF₂CF₂CF₂O)_{w4}(CF₂CF₂CF₂CF₂O)_{w5}(CF₂)_{w6}— (Rf)

(In Formula (Rf), w2, w3, w4, and w5 indicate an average degree of polymerization and each independently represent, 0 to 30, provided that all of w2, w3, w4, and w5 are not 0 at the same time; w1 and w6 are average values indicating the number of —CF₂ and each independently represents 1 to 3, and the arrangement sequence of repeating units in Formula (Rf) is not particularly limited.)

In Formula (Rf), w2, w3, w4, and w5 indicate an average degree of polymerization and each independently represents 0 to 30, preferably 0 to 20, more preferably 0 to 15.

In Formula (Rf), w1 and w6 are average values indicating the number of —CF₂— and each independently represents 1 to 3, w1 and w6 are determined according to the structure or the like of the repeating units arranged at the end portions of the chain structure in the polymer represented by Formula (Rf).

(CF₂O), (CF₂CF₂O), (CF₂CF₂CF₂O), and (CF₂CF₂CF₂CF₂O) in Formula (Rf) are repeating units. The arrangement sequence of the repeating units in Formula (Rf) are not particularly limited. In addition, the number of types of repeating units in Formula (Rf) are not particularly limited.

The PFPE chains preferably have, for example, a structure represented by Formula (Rf-1) below.

—(CF₂)_{w7}O—(CF₂CF₂O)_{w8}—(CF₂CF₂CF₂O)_{w9}—(CF₂)_{w10}— (Rf-1)

(In Formula (Rf-1), w8 and w9 indicate an average degree of polymerization and each independently represents 0.1 to 30, and w7 and w10 are average values indicating the number of —CF₂— and each independently represents 1 to 2.)

The arrangement sequence of (CF₂CF₂O) and (CF₂CF₂CF₂O) which are repeating units in Formula (Rf-1) is not particularly limited. Formula (Rf-1) may include any of a random copolymer, a block copolymer, and an alternating copolymer composed of the monomer units (CF₂CF₂O) and (CF₂CF₂CF₂O). In Formula (Rf-1), w8 and w9 indicating an average degree of polymerization each independently represents 0.1 to 30, preferably 0.1 to 20, more preferably 1 to 15. In Formula (Rf-1), w7 and w10 are average values indicating the number of —CF₂— and each independently represents 1 to 2. w7 and w10 are determined according to the structure or the like of the repeating units arranged at the end portions of the chain structure in the polymer represented by Formula (Rf-1).

R² and R⁴ are preferably any one represented by Formulae (4) to (6) below. In the case where R² and R⁴ are any of Formulae (4) to (6), a fluorine-containing ether compound is easily synthesized, which is preferable.

In addition, in the case where R² and R⁴ are any of Formulae (4) to (6), the ratio of the number of oxygen atoms (the number of ether bonds (—O—)) to the number of carbon atoms in the perfluoropolyether chain is appropriate. For this reason, a fluorine-containing ether compound with moderate hardness is obtained. Accordingly, a fluorine-containing ether compound applied onto a protective layer is less likely to be aggregated on the protective layer, and a lubricating layer having an even thinner thickness at a sufficient coating rate can be formed. In addition, in the case where R² and R⁴ are any of Formulae (4) to (6), a fluorine-containing ether compound is obtained with which a lubricating layer having favorable resistance to chemical substances is obtained.

—CF₂O—(CF₂CF₂O)_{b}—(CF₂O)ₑ—CF₂— (4)
(b and c in Formula (4) indicate an average degree of polymerization, each independently representing 0 to 30, provided that b and c are not 0 at the same time.)

The arrangement sequence of (CF₂—CF₂—O) and (CF₂—O) which are repeating units in Formula (4) is not particularly limited. In Formula (4), the number b of (CF₂—CF₂—O) and the number c of (CF₂—O) may be the same as or different from each other. However, b and c are not 0 at the same time. Formula (4) may include any of a random copolymer, a block copolymer, and an alternating copolymer composed of the monomer units (CF₂—CF₂—O) and (CF₂—O).

In a case where R² and/or R⁴ in Formula (1) are Formula (4), b indicating an average degree of polymerization is 0 to 30, preferably 1 to 20, and more preferably 1 to 15, b may be 1 to 10 or 1 to 5. In the case where R² and/or R⁴ in Formula (1) are Formula (4), c indicating an average degree of polymerization is 0 to 30, preferably 0 to 20, and more preferably 0 to 15. c may be 1 to 10 or 1 to 5. In addition, in a case where c is 0, b is preferably 1 to 17.

—CF(CF₃)—(OCF(CF₃)CF₂)_{d}—OCF(CF₃)— (5)
(d in Formula (5) indicates an average degree of polymerization and represents 0.1 to 30.)

In Formula (5), in a case where d indicating an average degree of polymerization is 0.1 to 30, the number-average molecular weight of the fluorine-containing ether compound of the present, embodiment is likely to fall within a preferred range. d is preferably 1 to 30, more preferably 2 to 20, and still more preferably 3 to 10.

—CF₂CF₂O—(CF₂CF₂CF₂O)ₑ—CF₂CF₂— (6)
(e in Formula (6) indicates an average degree of polymerization and represents 0.1 to 30.)

In Formula (6), in a case where e indicating an average degree of polymerization is 0.1 to 30, the number-average molecular weight of the fluorine-containing ether compound of the present embodiment is likely to fall within a preferred range. e is preferably 1 to 20, more preferably 2 to 15, and still more preferably 2 to 8.

(Terminal Groups Represented by R¹ and R⁵)

R¹ and R⁵ in the fluorine-containing ether compound represented by Formula (I) are terminal groups respectively containing two or three polar groups, in which individual polar groups are bound to different carbon atoms and the carbon atoms to which the polar groups are bound are bound to each other via a linking group containing a carbon atom to which the polar groups are not bound. The terminal groups represented by R¹ and R⁵ preferably do not contain perfluoropolyether chains (PFPE chains).

Since the fluorine-containing ether compound represented by Formula (1) has two or three polar groups contained in R¹ and R⁵, a lubricating layer which has a high coating rate and excellent adhesion properties with respect to a protective layer can be formed. With respect to the number of the polar group in R¹ and R⁵, it is preferable that R¹ and R⁵ each contain two polar groups to obtain a fluorine-containing ether compound with which a lubricating layer having even more favorable resistance to chemical substances is obtained. If R¹ and R⁵ contain too many polar groups, the polarity of the fluorine-containing ether compound becomes excessively high, resulting in low fluidity, and the wear resistance of a lubricating layer containing the fluorine-Containing ether compound is likely to deteriorate. In the present embodiment, since the number of the polar group in R¹ and R⁵ is two or three respectively, the deterioration in wear resistance due to excessively high polarity of the fluorine-containing ether compound can be suppressed.

Examples of the two or three polar groups in the terminal groups represented by R¹ and R⁵ include a hydroxyl group (—OH), an amino group (—NH₂), a carboxy group (—COOH), and a mercapto group (—SH). An ether bond (—O—) is not considered as the polar groups in R¹ and R⁵. Among the above-described polar groups, it is particularly preferable that the polar groups be hydroxyl groups. The two or three polar groups contained in the terminal group represented by R¹ may be different from each other, or all may be the same. The two or three polar groups contained in the terminal group represented by R may be different from each other, or all may be the same. All of the two or three polar groups in the terminal groups represented by R¹ and R⁵ are preferably hydroxyl groups.

A hydroxyl group has a strong interaction with a protective layer of a magnetic recording medium, particularly a protective layer made of a carbon-based material. Accordingly, it is preferable that some or all of the two or three polar groups in the terminal groups represented by R¹ and R⁵ be hydroxyl groups because a lubricating layer containing the fluorine-containing ether compound has even higher adsorption power with respect to a protective layer.

The terminal groups represented by R¹ and R⁵ preferably contain an ether bond. Furthermore, in the terminal groups represented by R¹ and R⁵, it is preferable that the two or three polar groups be bound to different carbon atoms, and the carbon atoms to which the polar groups are bound be bound to each other via a linking group containing an oxygen atom (—O— (ether bond)) and a carbon atom to which the polar groups are not bound. The linking group containing an ether bond imparts flexibility to the molecular structure of the fluorine-containing ether compound having the terminal groups represented by R¹ and R⁵. In a case of a fluorine-containing ether compound in which the carbon atoms to which the polar groups are bound are bound to each other via a linking group containing an ether bond and a carbon atom to which the polar groups are not bound, a lubricating layer containing the fluorine-containing ether compound is likely to be adsorbed onto a protective layer and the adhesion properties between the lubricating layer and the protective layer are excellent compared to, for example, a fluorine-containing ether compound in which two polar groups contained in terminal groups are bound to different carbon atoms and the carbon atoms to which the polar groups are bound are directly bound to each other.

The terminal groups represented by R¹ and R⁵ in Formula (1) can be appropriately selected depending on the performance required of a lubricant containing a fluorine-containing ether compound.

R¹ and R⁵ may be the same as or different from each other. In addition, the number of polar groups contained in the terminal group represented by R¹ and the number of polar groups contained in the terminal group represented by R⁵ may be the same as or different from each other. In a case where R¹ and R³ are the same terminal groups, a fluorine-containing ether compound is easily synthesized, which is preferable.

R¹ and R⁵ in Formula (1) are preferably terminal groups of any of Formulae (7) to (10) below. If R¹ and R⁵ are terminal groups of any of Formulae (7) to (10), the coating rate and the adhesion properties between the protective layer, on which a lubricant containing the fluorine-containing ether compound of the present embodiment is applied, and the lubricating layer formed through application of the lubricant are improved.

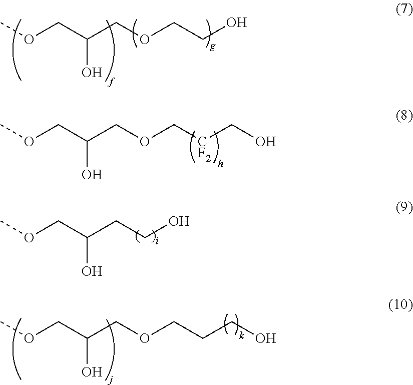

(In Formula (7), f represents an integer of 1 to 2, and g represents an integer of 1 to 5.)

(In Formula (8), h represents an integer of 1 to 5.)

(In Formula (9), i represents an integer of 1 to 5.)

(In Formula (10), j represents an integer of 1 to 2, and k represents an integer of 1 to 2.)

In Formula (7), f represents an integer of 1 to 2. f is preferably 2 from the viewpoint of adhesion properties between the lubricating layer and the protective layer.

In Formula (7), g represents an integer of 1 to 5. If g is an integer of 1 to 5, the distance between hydroxyl groups in the terminal group represented by Formula (7) becomes appropriate, and a fluorine-containing ether compound with which a lubricating layer having a high coating rate and excellent adhesion properties with respect to a protective layer can be formed is obtained. g is preferably 1 or 2 and most preferably 1 from the viewpoint of adhesion properties between the lubricating layer and the protective layer.

In Formula (8), h represents an integer of 1 to 5. If h is an integer of 1 to 5, the distance between hydroxyl groups on the R² or R⁴ side and the hydroxyl group in the terminal end becomes appropriate, and a fluorine-containing ether compound with which a lubricating layer having a high coating rate and excellent adhesion properties with respect to a protective layer can be formed is obtained. h is preferably 1 to 3 and most preferably 1 from the viewpoint of adhesion properties between the lubricating layer and the protective layer. Since the terminal group represented by Formula (8) contains —CF₂—, lubricity is imparted to the lubricating layer containing the fluorine-containing ether compound. For this reason, a lubricating layer having superior wear resistance can be formed with the fluorine-containing ether compound having the terminal group represented by Formula (8).

In Formula (9), i represents an integer of 1 to 5. If i is an integer of 1 to 5, the distance between hydroxyl groups on the R² or R⁴ side and the hydroxyl group in the terminal end becomes appropriate, and a fluorine-containing ether compound with which a lubricating layer having a high coating rate and excellent adhesion properties with respect to a protective layer can be formed is obtained. i is preferably 1 or 2 and most preferably 1 from the viewpoint of adhesion properties between the lubricating layer and the protective layer.

In Formula (10), j represents an integer of 1 to 2. j is preferably 2 from the viewpoint of adhesion properties between the lubricating layer and the protective layer.

In Formula (10), k represents an integer of 1 to 2. If k is an integer of 1 to 2, the distance between hydroxyl groups in the terminal group represented by Formula (10) becomes appropriate, and a fluorine-containing ether compound with which a lubricating layer having a high coating rate and excellent adhesion properties with respect to a protective layer can be formed is obtained. k is preferably 1 from the viewpoint of adhesion properties between the lubricating layer and the protective layer. In addition, k is preferably 2 from the viewpoint of wear resistance.

In addition, in the fluorine-containing ether compound of the present embodiment, one or more polar groups contained in R³ and two or three polar groups which are arranged at appropriate distances and contained in each of R¹ and R⁵ are arranged in the entire molecule with a good balance. For this reason, the lubricating layer containing the fluorine-containing ether compound of the present embodiment has excellent adhesiveness (adhesion properties) with respect to the protective layer and can cover the surface of the protective layer with a high coating rate. For this reason, the lubricating layer containing the fluorine-containing ether compound of the present embodiment has favorable resistance to chemical substances, can be made thinner, and can contribute to reduction in magnetic spacing in a magnetic recording medium.

It is preferable that the fluorine-containing ether compound represented by Formula (1) be specifically any compound represented by Formulae (A) to (P) below.

Since repeating numbers indicated by ba1 to bh1, ba2 to bh2, ca1 to ch1, and ca2 to ch2 in Formulae (A) to (H), bi1 and bi2 in Formula (1), ej1 and ej2 in Formula (J), and bk1 to bp1, bk2 to bp2, ck1 to cp1, and ck2 to cp2 in Formulae (K) to (P) are all values, indicating average degrees of polymerization, they are not necessarily integers.

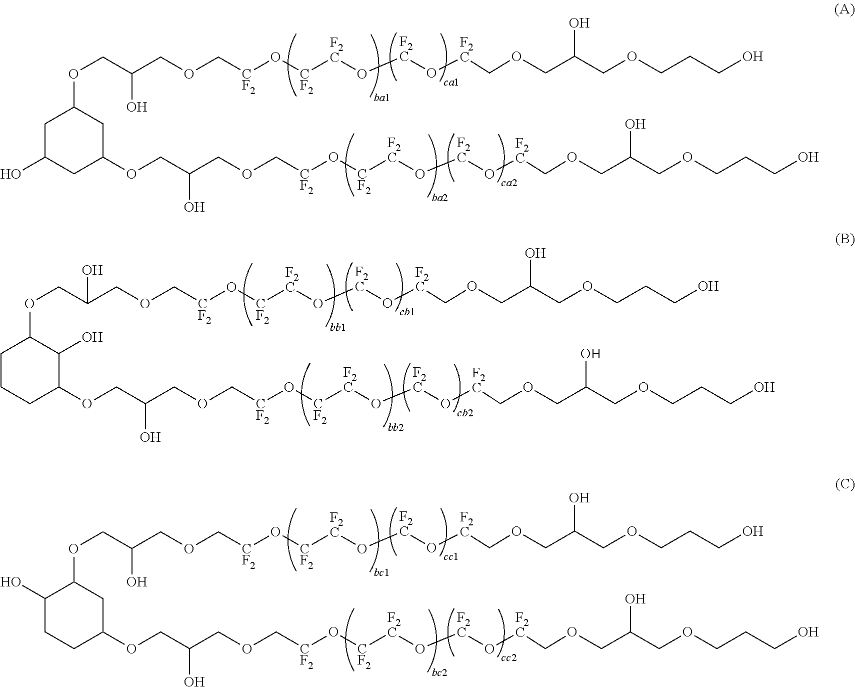

(In Formula (A), ba1, ca1, ba2, and ca2 indicate an average degree of polymerization, ba1 and ba3 represent 0 to 30, and ca1 and ca2 represent 0 to 30, provided that ba1 and ca1 are not 0 at the same time and ba2 and ca2 arm not 0 at the same time.)

(In Formula (8), bb1, cb1, bb2, and cb1 indicate an average degree of polymerization, bb1 and bb2 represent 0 to 30, and cb1 and cb2 represent 0 to 30, provided that bb1 and cb1 are not 0 at the same time and bb2 and cb2 are not 0 at the same time.)

(In Formula (C), bc1, cc1, bc2, and cc2 indicate an average degree of polymerization, bc1 and bc2 represent 0 to 30, and cc1 and cc2 represent 0 to 30, provided that bc1 and cc1 are not 0 at the same time and bc2 and cc2 am not 0 at the same time.)

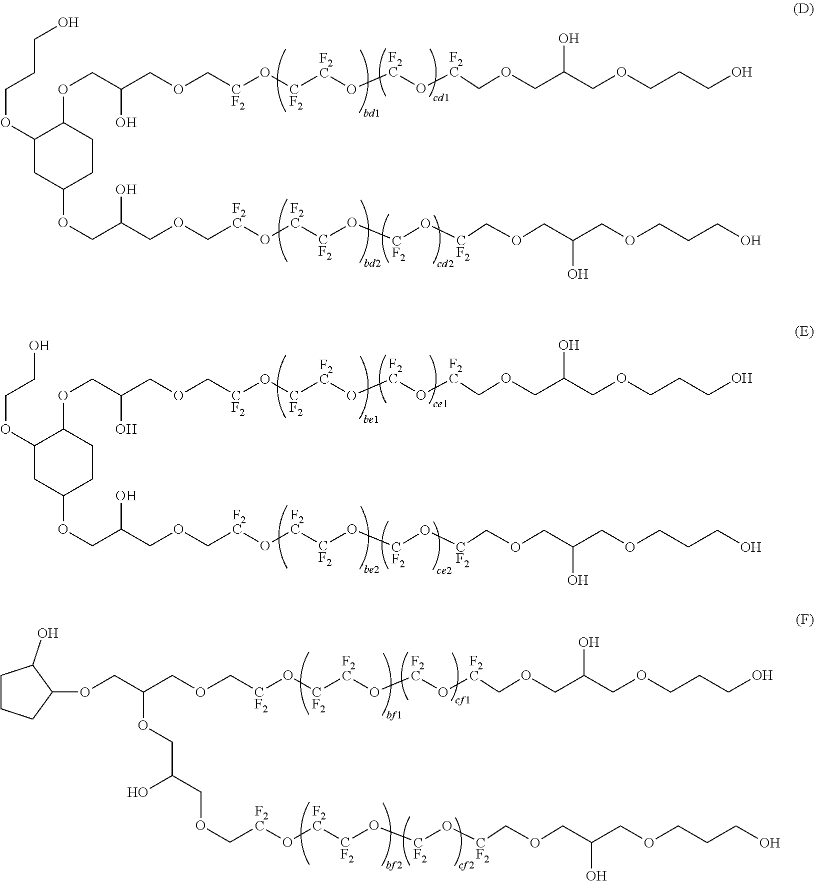

(In Formula (D), bd1, cd1, bd2, and cd2 indicate an average degree of polymerization, bd1 and bd2 represent 0 to 30, and cd1 and cd2 represent 0 to 30, provided that bd1 and cd1 are not 0 at the same time and bd2 and cd2 are not 0 at the same time.)

(In Formula (E), be1, ce1, be2, and ce2 indicate an average degree of polymerization, be1 and be2 represent 0 to 30, and ce1 and ce2 represent 0 to 30, provided that be1 and ce1 are not 0 at the same time and be2 and ce2 are not 0 at the same time.)

(In Formula (F), bf1, cf1, bf2, and cf2 indicate an average degree of polymerization, bf1 and bf2 represent 0 to 30, and cf1 and cf2 represent 0 to 30, provided that bf1 and cf1 are not 0 at the same time and bf2 and cf2 are not 0 at the same time.)

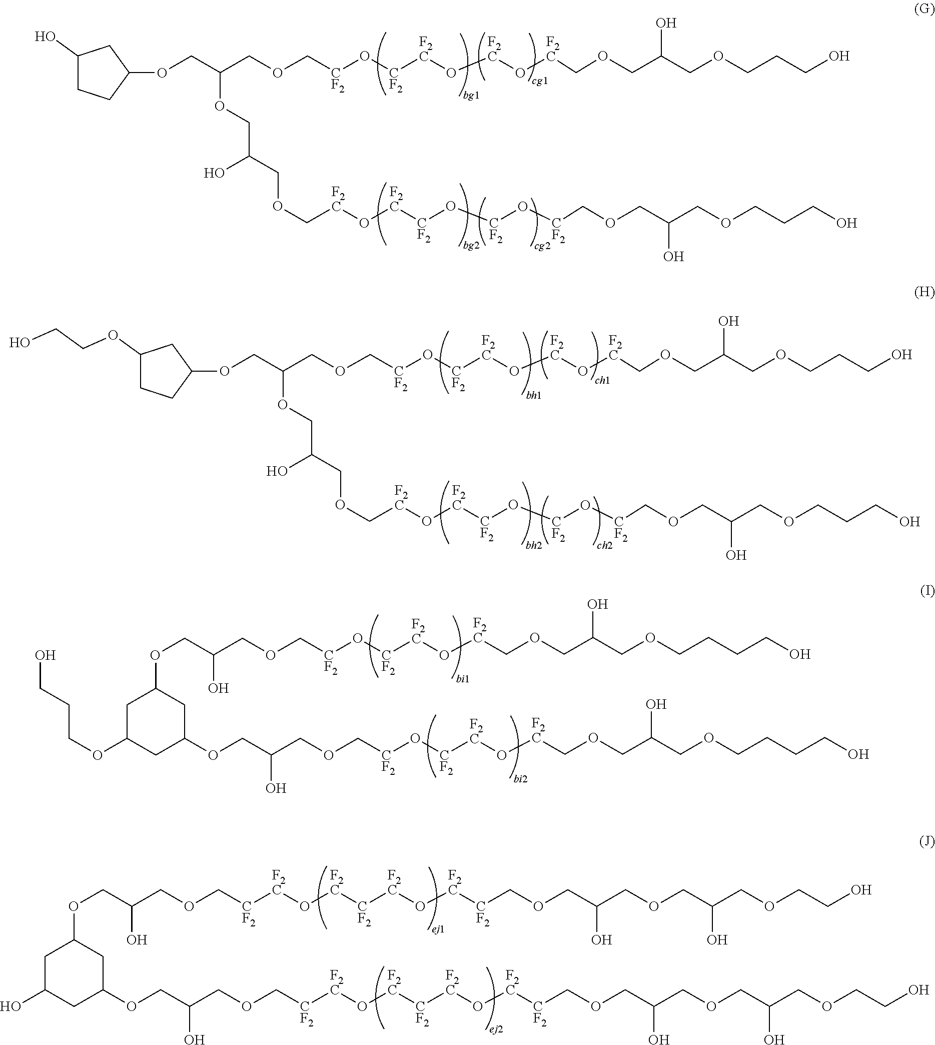

(In Formula (G), bg1, cg1, bg2, and cg2 indicate an average degree of polymerization, bg1 and bg2 represent 0 to 30, and cg1 and cg2 represent 0 to 30, provided that bg1 and cg1 are not 0 at the same time and bg2 and cg2 are not 0 at the same time.)

(In Formula (H), bh1, ch1, bh2, and ch2 indicate an average degree of polymerization, bh1 and bh2 represent 0 to 30, and ch1 and ch2 represent 0 to 30, provided that bh1 and ch1 are not 0 at the same time and bh2 and ch2 are not 0 at the same time.)

(In Formula (I), bi1 and bi2 indicate an average degree of polymerization and represent 0.1 to 30.)

(In Formula (J), ej1 and ej2 indicate an average degree of polymerization and represent 0.1 to 30.)

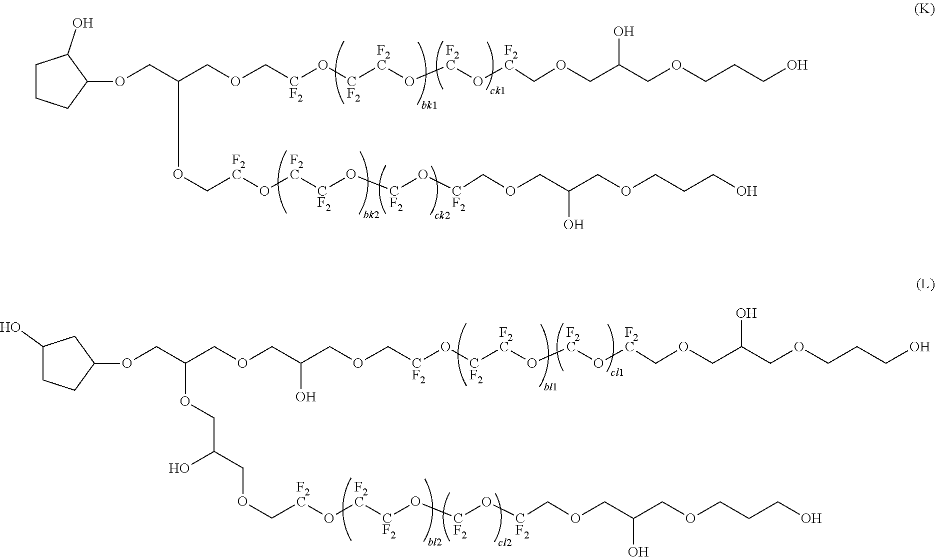

(In Formula (K), bk1, ck1, bk2, and ck2 indicate an average degree of polymerization, bk1 and bk2 represent 0 to 30, and ck1 and ck2 represent 0 to 30, provided that bk1 and ck1 are not 0 at the same time and bk2 and ck2 are not 0 at the same time.)

(In Formula (L), bl1, cl1, bl2, and cl2 indicate an average degree of polymerization, bl1 and bl2 represent 0 to 30, and cl1 and cl2 represent 0 to 30, provided that bl1 and cl1 are not 0 at the same time and bl2 and cl2 are not 0 at the same time.)

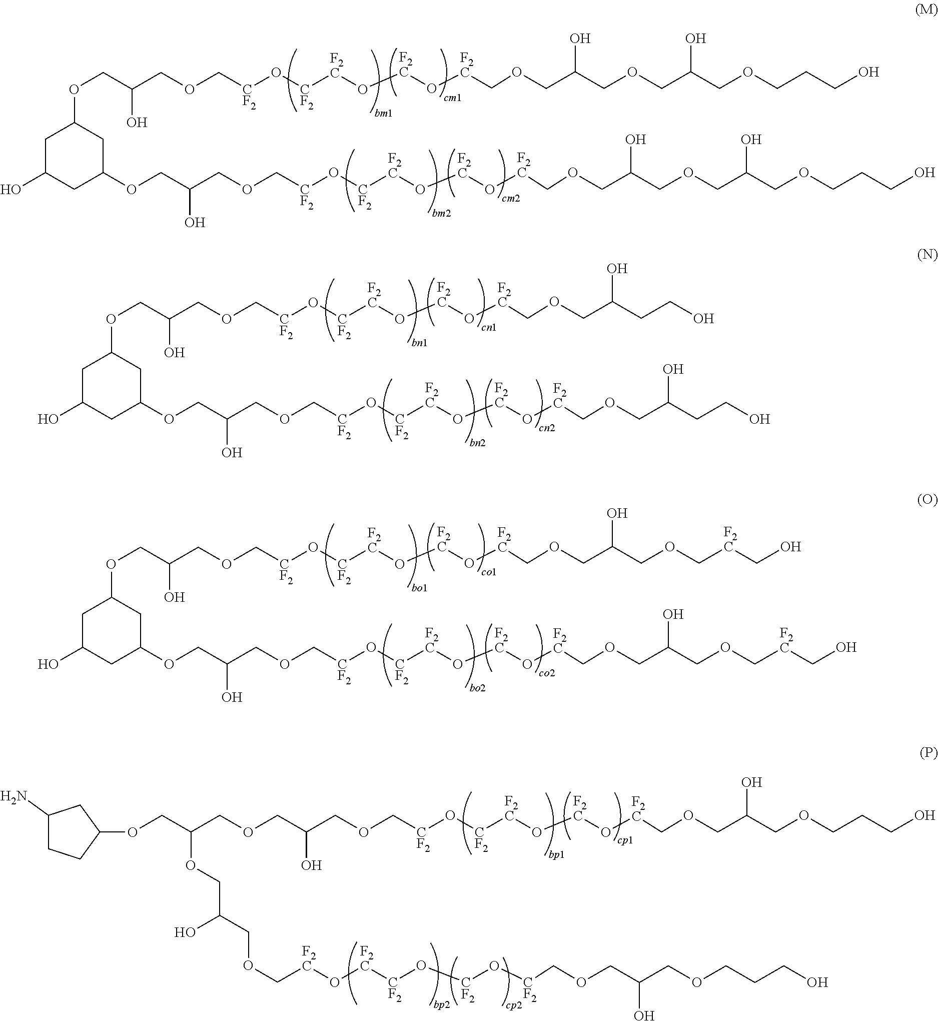

(In Formula (M), bm1, cm1, bm2, and cm2 indicate an average degree of polymerization, bm1 and bm2 represent 0 to 30, and cm1 and cm2 represent 0 to 30, provided that bm1 and cm1 are not 0 at the same time and bm2 and cm2 are not 0 at the same time.)

(In Formula (N), bn1, cn1, bn2, and cn2 indicate an average degree of polymerization, bn1 and bn2 represent 0 to 30, and cn1 and cn2 represent 0 to 30, provided that bn1 and cn1 are not 0 at the same time and bn2 and cn2 are not 0 at the same time.)

(In Formula (O), bo1, co1, bo2, and co2 indicate an average degree of polymerization, bo1 and bo2 represent 0 to 30, and co1 and co2 represent 0 to 30, provided that bo1 and co1 are not 0 at the same time and bo2 and co2 are not 0 at the same time.)

(In Formula (P), bp1, cp1, bp2, and cp2 indicate an average degree of polymerization, bp1 and bp2 represent 0 to 30, and cp1 and cp2 represent 0 to 30, provided that bp1 and cp1 are not 0 at the same time and bp2 and cp2 are not 0 at the same time.)

In all of the compounds represented by Formulae (A) to (E), (1) to (J), and (M) to (O), R³ in Formula (1) above is cyclohexane.

In all of the compounds represented by Formulae (F) to (H) (K) to (L), and (P), the alicyclic structure of R³ in Formula (I) above is cyclopentane.

In all of the compounds represented by Formulae (A) to (E), (I), (J), and (M) to (O), R³ in Formula (I) above is Formula (2-1). In the compounds represented by Formulae (F) to (H), R³ is Formula (2-3). In the compound represented by Formula (K), R³ is Formula (2-2). In the compounds represented by Formulae (L) and (P), R³ is Formula (2-4).

In all of the compounds represented by Formulae (A) to (H), (K) to (L), and (P), R¹ and R⁵ in Formula (1) above are represented by Formula (10), and j and k in Formula (10) are 1. In the compound represented by Formula (1). R¹ and R⁵ are represented by Formula (10), and j is 1 and k is 2 in Formula (10). In the compound represented by Formula (J), R¹ and R⁵ are represented by Formula (7), and f is 2 and g is 1 in Formula (7). In the compound represented by Formula (M), R¹ and R⁵ are represented by Formula (10), and j is 2 and k is 1 in Formula (10). In the compound represented by Formula (N), R¹ and R⁵ are represented by Formula (9), and i in Formula (9) is 1. In the compound represented by Formula (O), R¹ and R⁵ are represented by Formula (8), and h in Formula (8) is 1.

In all of the compounds represented by Formulae (A) to (I) and (K) to (P), R² and R⁴ in Formula (1) above are Formula (4). In the compound represented by Formula (J), R² and R⁴ in Formula (1) above are Formula (6).

In Formulae (A) to (H) and (K) to (P), ba1 to bh1, ba2 to bh2, bk1 to bp1, and bk2 to bp2 may be 0, 1 to 20, 1 to 10, or 1 to 5. In Formulae (A) to (H) and (K) to (P), ca1 to ch1, ca2 to ch2, ck1 to cp1, and ck2 to cp2 may be 0, 1 to 20, 1 to 10, or 1 to 5. In Formulae (I) and (J), bi1, bi2, cj1, and ej2 may be 1 to 20, 1 to 10, or 1 to 5.

If the fluorine-containing ether compound represented by Formula (1) is any compound represented by Formulae (A) to (P) above, the procurement of raw materials is easy and a lubricating layer having superior resistance to chemical substances and wear resistance can be formed even if the lubricating layer is thin, which is preferable.

In the fluorine-containing ether compound of the present embodiment, the number-average molecular weight (Mn) of the compound is preferably within a range of 500 to 10,000, more preferably within a range of 700 to 7,000, and particularly preferably within a range of 1,000 to 3,000. If the number-average molecular weight is 500 or more, a lubricant containing the fluorine-containing ether compound of the present embodiment hardly evaporates, whereby the lubricant can be prevented from evaporating and transferring to a magnetic head. In addition, if the number-average molecular weight is 10,000 or less, the fluorine-containing ether compound has an appropriate viscosity, and a thin lubricating layer can be easily formed by applying a lubricant containing this fluorine-containing ether compound. If the number-average molecular weight is 3,000 or less, in a case where the fluorine-containing ether compound is applied to a lubricant, the viscosity of the lubricant becomes appropriate for handling, which is more preferable.

The number-average molecular weight (Mn) of the fluorine-containing ether compound is a value measured by ¹H-NMR and ¹⁹F-NMR with AVANCE 11400 manufactured by Bruker BioSpin Group. In the nuclear magnetic resonance (NMR) measurement, a sample is diluted with a single or mixed solvent of hexafluorobenzene, acetone-d, tetrahydrofuran-d, and the like and used in the measurement. As the reference of the ¹⁹F-NMR chemical shift, the peak of hexafluorobenzene was set to −164.7 ppm, and as the reference of the ¹H-NMR chemical shift, the peak of acetone was set to 2.2 ppm.

“Production Method”

A method for producing the fluorine-containing ether compound of the present embodiment is not particularly limited, and the fluorine-containing ether compound can be produced using a well-known conventional production method. The fluorine-containing ether compound of the present embodiment can be produced using, for example, a production method shown below.

First, an alcohol represented by Formula (1-1) below corresponding to R¹—CH₂-R²—CH₂— in Formula (1) and an alcohol represented by Formula (1-2) below corresponding to —CH₂-R⁴—CH₂-R⁵ in Formula (1) are synthesized.

R¹—CH₂-R²—CH₂—OH (1-1)

(In Formula (1-1), R¹ and R² are the same as those in Formula (1).)

HO—CH₂-R⁴—CH₂—R⁵ (1-2)

(In Formula (1-2), R⁴ and R⁵ are the same as those in Formula (1).)

The alcohol represented by Formula (1-1) can be synthesized through a method of adding an epoxide compound having a structure corresponding to R¹ to a perfluoropolyether compound represented by HO—CH₂-R²—CH₂—OH to cause a reaction.

The alcohol represented by Formula (1-2) can be synthesized through a method of adding an epoxide compound having a structure corresponding to R to a perfluoropolyether compound represented by HO—CH₂—R—CH₂—OH to cause a reaction.

In a case of producing a fluorine-containing ether compound in which R³ in Formula (1) is Formula (2-1), an epoxide represented by Formula (1-3) below corresponding to X—[Y—CH₂—CH(OH)CH₂O-]₂ is synthesized.

X—{Y—CH₂-Ep}₂ (1-3)

(In Formula (1-3), Ep represents an epoxy group, and X and Y are the same as those in Formula (2-1).)

Next, the alcohols of Formulae (1-1) and (1-2) are added to the epoxide represented by Formula (1-3) to cause a reaction. As a result, a compound represented by Formula (I) is produced.

The epoxide represented by Formula (1-3) can be produced, for example, through a method of adding epibromohydrin to an alcohol having an alicyclic structure corresponding to R³ in Formula (1) to cause a reaction.

Specifically, in a case where, for example, an alicyclic structure corresponding to R¹ in Formula (1) is cyclohexane, an epoxide in which X in Formula (1-3) is cyclohexane having one hydroxyl group as a substituent and Y in Formula (1-3) is —O— can be produced through adding epibromohydrin to cyclohexanetriol to cause a reaction.

In a case of producing a fluorine-containing ether compound in which R³ in Formula (1) is Formula (2-2), an epoxide represented by Formula (1-4) below corresponding to X′—Y—CH₂—CH(O—)CH₂O— is synthesized.

X′—Y—CH₂-Ep (1-4)

(In Formula (1-4), Ep represents an epoxy group, and X⁺ and Y are the same as those in Formula (2-2).)

The epoxide represented by Formula (1-4) can be produced, for example, through a method of adding epibromohydrin to an alcohol having an alicyclic structure corresponding to R³ in Formula (1) to cause a reaction.

Specifically, in a case where, for example, an alicyclic structure corresponding to R¹ in Formula (1) is cyclohexane, an epoxide in which X′ in Formula (1-4) is cyclohexane having one hydroxyl group as a substituent and Y in Formula (1-4) is —O— can be produced through adding epibromohydrin to cyclohexanediol to cause a reaction.

Next, the alcohol of Formula (1-1) is added to the epoxide represented by (1-4) to cause a reaction. As a result, the compound represented by Formula (1-5) below is produced.

X′—Y—CH₂CH(OH)CH₂O—CH₂-R²—CH₂-R¹ (1-5)

(In Formula (1-5), X′ and Y are the same as those in Formula (2-2), and R¹ and R² are the same as those in Formula (1).)

Next, the hydroxyl group of the compound represented by Formula (1-5) is converted into a leaving group such as a bromo group or a methanesulfonic acid group, and the alcohol represented by Formula (1-2) is added to the compound to cause a reaction. As a result, a compound represented by Formula (1) is produced.

In a case of producing a fluorine-containing ether compound in which R³ in Formula (1) is Formula (2-3), the compound represented by Formula (1-5) (the compound in which X′ in Formula (1-5) is X) is produced similarly to the case where R³ is Formula (2-2).

Next, epibromohydrin is added to the hydroxyl group of the compound represented by Formula (1-5) (the compound in which X′ in Formula (1-5) is X) to cause a reaction to synthesize an epoxide represented by Formula (1-6).

X—Y—CH₂CH(OCH₂Ep)CH₂O—CH₂-R²—CH₂-R¹ (1-6)

(In Formula (1-6), Ep represents an epoxy group, X and Y are the same as those in Formula (2-3), and R¹ and R² are the same as those in Formula (1).)

Next, the alcohol represented by Formula (1-2) is added to the epoxide represented by Formula (1-6) to cause a reaction. As a result, a compound represented by Formula (1) is produced.

In a case of producing a fluorine-containing ether compound in which R³ in Formula (1) is Formula (2-4), an epoxide represented by Formula (1-7) below corresponding to X—Y—CH₂CH(OCH₂CH(OH)CH₂O—)CH₂OCH₂CH(OH)CH₂O— is synthesized.

X—Y—CH₂CH(OCH₂Ep)CH₂OCH₂Ep (1-7)

(In Formula (1-7). Ep represents an epoxy group, and X and Y are the same as those in Formula (2-4).)

Next, the alcohol; represented by Formulae (1-1) and (1-2) are added to the epoxide represented by Formula (1-7) to cause a reaction. As a result, a compound represented by Formula (1) is produced.

The epoxide represented by Formula (1-7) can be produced, for example, through the method shown below. An epoxide is obtained by adding epibromohydrin to an alcohol having an alicyclic structure corresponding to R in Formula (I) to cause a reaction. Next, allyl alcohol and allyl bromide are added to the obtained epoxide to cause a reaction, and the allyl groups are oxidized with meta-chloroperoxybenzoic acid to obtain the epoxide represented by Formula (1-7).

The produced fluorine-containing ether compound represented by Formula (1) is preferably purified, for example, through a method using column chromatography.

The fluorine-containing ether compound represented by Formula (1) is obtained through the above-described method.

The fluorine-containing ether compound of the present invention is the compound represented by Formula (1) above. Accordingly, when a lubricant containing this compound is used to form a lubricating layer on a protective layer, due to PFPE chains represented by R² and R⁴ in Formula (1), the surface of the protective layer is covered and frictional force between a magnetic head and the protective layer is reduced. In addition, in the lubricating layer formed with the lubricant containing the fluorine-containing ether compound of the present embodiment, the alicyclic structure in the organic group represented by R³ contributes to fluidity of the molecular structure of the fluorine-containing ether compound. For this reason, the alicyclic structure portion can be partially lifted from the protective layer. Accordingly, before the magnetic head directly collides with the protective layer, the lubricating layer collides with the magnetic head to protect the protective layer. Due to this function, the lubricating layer formed with the lubricant containing the fluorine-containing ether compound of the present embodiment has excellent wear resistance.

In addition, the lubricating layer containing the fluorine-containing ether compound of the present embodiment adheres closely to the protective layer through bonding between the protective layer and one or more polar groups contained in R³ in the fluorine-containing ether compound and bonding between the protective layer and two or three polar groups contained in each of the terminal groups represented by R¹ and R⁵.

In addition, in the fluorine-containing ether compound of the present embodiment, the two or three polar groups contained in the terminal groups represented by R¹ and R⁵ are bound to different carbon atoms, and the carbon atoms to which the polar groups are bound are bound to each other via a linking group containing a carbon atom to which the polar groups are not bound. For this reason, the fluorine-containing ether compound of the present embodiment is less likely to aggregate on the protective layer. Accordingly, the lubricating layer containing the fluorine-containing ether compounds of the present embodiment has a sufficient coating rate and favorable adhesion properties with respect to the protective layer.

From the above, according to the fluorine-containing ether compounds of the present embodiment, a lubricating layer which has excellent resistance to chemical substances and wear resistance and is firmly bound to the protective layer can be obtained.

[Lubricant for Magnetic Recording Medium]

A lubricant for a magnetic recording medium of the present embodiment contains the fluorine-containing ether compound represented by Formula (1).

The lubricant of the present embodiment can be used after being mixed as necessary with a well-known material used for lubricants within the scope not impairing the characteristics due to the incorporation of the fluorine-containing ether compound represented by Formula (1).

Specific examples of well-known materials include FOMBLIN (registered trademark) ZDIAC, FOMBLIN ZDEAL, and FOMBLIN AM-2001 (all manufactured by Solvay Solexis), and Moresco A20H (manufactured by Moresco Corporation). The number-average molecular weight of the well-known material used by being mixed with the lubricant of the present embodiment is preferably 1.000 to 10,000.

In a case where the lubricant of the present embodiment contains a material other than the fluorine-containing ether compound represented by Formula (1), the content of the fluorine-containing ether compound represented by Formula (1) in the lubricant of the present embodiment is preferably 50 mass % or more and more preferably 70 mass % or more. The content of the fluorine-containing ether compound represented by Formula (1) may be 80 mass % or more or 90 mass % or more. The upper limit of the content of the fluorine-containing ether compound represented by Formula (1) can be arbitrarily selected, and can be set to, for example, 99 mass % or less, and may be 95 mass % or less or 90 mass % or less.

Since the lubricant of the present embodiment contains the fluorine-containing ether compound represented by Formula (1), it can cover the surface of the protective layer with a high coating rate and can form the lubricating layer having excellent adhesion properties with respect to the protective layer even if the lubricating layer is thin. In addition, in the lubricant of the present embodiment, the alicyclic structure portion contained in R³ in the fluorine-containing ether compound represented by Formula (1) is partially lifted from the protective layer, thereby protecting the protective layer. Thus, according to the lubricant of the present embodiment, a lubricating layer which has excellent resistance to chemical substances and wear resistance can be obtained even if the lubricating layer is thin.

[Magnetic Recording Medium]

A magnetic recording medium of the present embodiment includes at least a magnetic layer, a protective layer, and a lubricating layer sequentially provided on a substrate.

In the magnetic recording medium of the present embodiment, one or more underlayers can be provided as necessary between the substrate and the magnetic layer. In addition, it is also possible to provide an adhesive layer and/or a soft magnetic layer between the underlayer and the substrate.

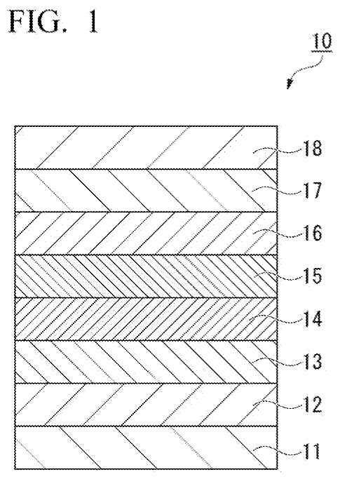

FIG. 1 is a schematic cross-sectional view showing an example of one embodiment of a magnetic recording medium of the present invention.

A magnetic recording medium 10 of the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

“Substrate”

As the substrate 11, for example, a non-magnetic substrate or the like in which a NiP or NiP alloy film is formed on a base made of metal or alloy material such as Al or an Al alloy can be used.

In addition, as the substrate 11, a non-magnetic substrate made of a non-metal material such as glass, ceramics, silicon, silicon carbide, carbon or a resin may be used, and a non-magnetic substrate in which a NiP or NiP alloy film is formed on a base made of this non-metal material may also be used.

“Adhesive Layer”

The adhesive layer 12 prevents the progress of corrosion of the substrate 11 which may occur in a case where the substrate 11 and the soft magnetic layer 13, which is provided on the adhesive layer 12, are arranged in contact with each other.

The material of the adhesive layer 12 can be appropriately selected from, for example, Cr, a Cr alloy, Ti, a Ti alloy, CrTi, NiAl, and an AlRu alloy. The adhesive layer 12 can be formed by, for example, a sputtering method.

“Soft Magnetic Layer”

The soft magnetic layer 13 preferably has a structure in which a first soft magnetic film, an interlayer made of a Ru film, and a second soft magnetic film are sequentially laminated. That is, the soft magnetic layer 13 preferably has a structure in which the interlayer made of a Ru film is sandwiched between the two soft magnetic films, whereby the soft magnetic films on and under the interlayer are antiferromagnetically coupled (AFC).

Examples of the material of the first soft magnetic film and the second soft magnetic film include a CoZrTa alloy and a CoFe alloy.

Any of Zr, Ta and Nb is preferably added to the CoFe alloy used for the first soft magnetic film and the second soft magnetic film. This accelerates the amorphization of the first soft magnetic film and the second soft magnetic film, makes it possible to improve the orientation of the first underlayer (seed layer) and makes it possible to reduce the flying height of a magnetic head.

The soft magnetic layer 13 can be formed by, for example, a sputtering method.

“First Underlayer”

The first underlayer 14 is a layer for controlling the orientations and crystal sizes of the second underlayer 15 and the magnetic layer 16 provided on the first underlayer 14.

Examples of the first underlayer 14 include a Cr layer, a Ta layer, a Ru layer, a CrMo alloy layer, a CoW alloy layer, a CrW alloy layer, a CrV alloy layer, and a CrTi alloy layer.

The first underlayer 14 can be formed by, for example, a sputtering method.

“Second Underlayer”

The second underlayer 15 is a layer that controls the orientation of the magnetic layer 16 to be favorable. The second underlayer 15 is preferably a Ru or Ru alloy layer.

The second underlayer 15 may be a single layer or may be composed of a plurality of layers. In a case where the second underlayer 15 is composed of a plurality of layers, all of the layers may be composed of the same material or at least one layer may be composed of a different material.

The second underlayer 15 can be formed by, for example, a sputtering method.

“Magnetic Layer”

The magnetic layer 16 is made of a magnetic film in which the easy magnetization axis is directed in a perpendicular or parallel direction with respect to the substrate surface. The magnetic layer 16 is a layer containing Co and Pt and may be a layer further containing an oxide, Cr, B, Cu, Ta, Zr, or the like in order to improve SNR characteristics.

Examples of the oxide contained in the magnetic layer 16 include SiO₂, SiO, Cr₂O₃, CoO, Ta₂O₃, and TiO₂.

The magnetic layer 16 may be composed of a single layer or may be composed of a plurality of magnetic layers made of materials with different compositions.

For example, in a case where the magnetic layer 16 is composed of three layers of a first magnetic layer, a second magnetic layer, and a third magnetic layer sequentially laminated from below, the first magnetic layer is preferably a granular structure made of a material containing Co, Cr, and Pt and further containing an oxide. As the oxide contained in the first magnetic layer, for example, oxides of Cr, Si, Ta, Al, Ti, Mg, Co, or the like are preferably used. Among them, in particular, TiO₂, Cr₂O₃, SiO₂, and the like can be suitably used. In addition, the first magnetic layer is preferably made of a composite oxide to which two or more oxides have been added. Among them, in particular, Cr₂O₃—SiO₂. Cr₂O₃—TiO₂, SiO₂—TiO₂, and the like can be suitably used.

The first magnetic layer may contain, in addition to Co. Cr. Pt, and the oxide, one or more elements selected from B, Ta, Mo, Cu, Nd, W, Nb, Sm, Tb, Ru, and Re.

For the second magnetic layer, the same material as for the first magnetic layer can be used. The second magnetic layer preferably has a granular structure.

The third layer preferably has a non-granular structure made of a material containing Co, Cr, and Pt but containing no oxides. The third magnetic layer may contain, in addition to Co. Cr, and Pt, one or more elements selected from B, Ta, Mo, Cu, Nd, W, Nb, Sm, Tb, Ru, Re, and Mn.

In a case where the magnetic layer 16 is formed of a plurality of magnetic layers, a non-magnetic layer is preferably provided between the magnetic layers adjacent to each other. In a case where the magnetic layer 16 is made tip of three layers of the first magnetic layer, the second magnetic layer and the third magnetic layer, it is preferable to provide a non-magnetic layer between the first magnetic layer and the second magnetic layer and a non-magnetic layer between the second magnetic layer and the third magnetic layer.

For the non-magnetic layer provided between the magnetic layers adjacent to each other in the magnetic layer 16, it is possible to suitably use, for example. Ru, a Ru alloy, a CoCr alloy, and a CoCrX1 alloy (X1 represents one or more elements selected from Pt, Ta, Zr, Re, Ru, Cu, Nb, Ni, Mn, Ge, Si, O, N, W, Mo, Ti, V, and B).

For the non-magnetic layer provided between the magnetic layers adjacent to each other in the magnetic layer 16, an alloy material containing an oxide, a metallic nitride or a metallic carbide is preferably used. Specifically, as the oxide, for example. SiO₂, Al₂O₃, Ta₂O₅, Cr₂O₃, MgO, Y₂O₃, and TiO₂ can be used. As the metallic nitride, for example, AlN, Si₃N₃, TaN, and CrN can be used. As the metallic carbide, for example, TaC, BC, and SiC can be used.

The non-magnetic layer can be formed by, for example, a sputtering method.

The magnetic layer 16 is preferably a magnetic layer for perpendicular magnetic recording in which the easy magnetization axis is directed in a direction perpendicular to the substrate surface in order to realize a higher recording density. The magnetic layer 16 may be a magnetic layer for in-plane magnetic recording.

The magnetic layer 16 may be formed by any well-known conventional method such as a deposition method, an ion beam sputtering method, or a magnetron sputtering method. The magnetic layer 16 is normally formed by a sputtering method.

“Protective Layer”

The protective layer 17 protects the magnetic layer 16. The protective layer 17 may be composed of a single layer or may be composed of a plurality of layers. As the material of the protective layer 17, carbon, nitrogen-containing carbon, silicon carbide, and the like can be exemplified.

As the protective layer 17, a carbon-based protective layer can be preferably used, and, in particular, an amorphous carbon protective layer is preferable. When the protective layer 17 is a carbon-based protective layer, an interaction with a polar group (particularly a hydroxyl group) contained in the fluorine-containing ether compound in the lubricating layer 18 is further enhanced, which is preferable.

The adhesive force between the carbon-based protective layer and the lubricating layer 18 can be controlled by forming the carbon-based protective layer with hydrogenated carbon and/or nitrogenated carbon and adjusting the hydrogen content and/or the nitrogen content in the carbon-based protective layer. The hydrogen content in the carbon-based protective layer is preferably 3 to 20 atomic % when measured by the hydrogen forward scattering method (HFS). In addition, the nitrogen content in the carbon-based protective layer is preferably 4 to 15 atomic % when measured by X-ray photoelectron spectroscopy (XPS).

The hydrogen and/or nitrogen contained in the carbon-based protective layer do not need to be uniformly contained throughout the entire carbon-based protective layer. The carbon-based protective layer is suitably formed as, for example, a composition gradient layer in which nitrogen is contained in the lubricating layer 18 side of the protective layer 17 and hydrogen is contained in the magnetic layer 16 side of the protective layer 17. In this case, the adhesive force between the magnetic layer 16 and the carbon-based protective layer and the adhesive force between the lubricating layer 18 and the carbon-based protective layer further improve.

The film thickness of the protective layer 17 is preferably set to 1 nm to 7 nm. When the film thickness of the protective layer 17 is 1 nm or more, performance as the protective layer 17 can be sufficiently obtained. The film thickness of the protective layer 17 is preferably 7 nm or less from the viewpoint of reducing the thickness of the protective layer 17.

As a method for forming the protective layer 17, it is possible to use a sputtering method in which a carbon-containing target material is used, a chemical vapor deposition (CVD) method in which a hydrocarbon raw material such as ethylene or toluene is used, an ion beam deposition (IBL) method, and the like.

In the case of forming a carbon-based protective layer as the protective layer 17, the carbon-based protective layer can be formed by, for example, a DC magnetron sputtering method. Particularly, in the case of forming a carbon-based protective layer as the protective layer 17, an amorphous carbon protective layer is preferably formed by a plasma CVD method. The amorphous carbon protective layer formed by the plasma CVD method has a uniform surface with small roughness.

“Lubricating Layer”

The lubricating layer 18 prevents contamination of the magnetic recording medium 10. In addition, the lubricating layer 18 reduces frictional force of a magnetic head of a magnetic recording/reproducing device, which slides on the magnetic recording medium 10, thereby improving the durability of the magnetic recording medium 10.

The lubricating layer 18 is formed in contact with the protective layer 17 as shown in FIG. 1. The lubricating layer 18 contains the above-described fluorine-containing ether compound.

In a case where the protective layer 17, which is placed below the lubricating layer 18, is a carbon-based protective layer, particularly, the lubricating layer 18 is bound to the protective layer 17 with a high binding force. As a result, the magnetic recording medium 10 in which the surface of the protective layer 17 is coated with the lubricating layer 18 at a high coating rate in spite of a thin thickness is likely to be obtained, and contamination on the surface of the magnetic recording medium 10 can be effectively prevented.

The average film thickness of the lubricating layer 18 can be arbitrarily selected, and is preferably 0.5 nm (5 Å) to 2.0 nm (20 Å) and more preferably 0.5 nm (5 Å) to 1.0 nm (10 Å). When the average film thickness of the lubricating layer 18 is 0.5 nm or more, the lubricating layer 18 does not have an island shape or a mesh shape and is formed in a uniform film thickness. For this reason, the surface of the protective layer 17 can be coated with the lubricating layer 18 at a high coating rate. In addition, when the average film thickness of the lubricating layer 18 is set to 2.0 nm or less, it is possible to sufficiently reduce the thickness of the lubricating layer 18 and to sufficiently decrease the flying height of a magnetic head.

In a case where the surface of the protective layer 17 is not sufficiently coated with the lubricating layer 18 at a high coating rate, an environmental substance adsorbed to the surface of the magnetic recording medium 10 passes through voids in the lubricating layer 18 and intrudes into the layer below the lubricating layer 18. The environmental substance that has intruded into the underlayer of the lubricating layer 18 is adsorbed and bound to the protective layer 17 and generates a contamination substance. At the time of reproducing magnetic records, this contamination substance (aggregated component) adheres (transfers) to a magnetic head as a smear to break the magnetic head or degrade the magnetic recording/reproducing characteristics of magnetic recording/reproducing devices.

Examples of the environmental substance that generates the contamination substance include siloxane compounds (cyclic siloxane and linear siloxane), ionic impurities, hydrocarbons having a relatively high molecular weight such as octacosane, and plasticizers such as dioctyl phthalate. Examples of metal ions contained in the ionic impurities include a sodium ion and a potassium ion. Examples of inorganic ions contained in the ionic impurities include a chlorine ion, a bromine ion, a nitrate ion, a sulfate ion, and an ammonium ion. Examples of organic ions contained in the ionic impurities include an oxalate ion and a formate ion.

“Method for Forming Lubricating Layer”

Examples of methods for forming the lubricating layer 18 include a method in which a magnetic recording medium that is not yet fully manufactured and thus includes the individual layers up to the protective layer 17 formed on the substrate 1 is prepared and a solution for forming a lubricating layer is applied onto the protective layer 17 and dried.

The solution for forming a lubricating layer can be obtained, for example, by dispersing and dissolving the above-described lubricant for a magnetic recording medium of the embodiment in a solvent as necessary and adjusting the viscosity and concentration to be suitable for application methods.

Examples of solvents used for the solution for forming a lubricating layer include fluorine-based solvents such as VERTREL (registered trademark) XF (trade name, manufactured by Dupont-Mitsui Fluorochemicals Co., Ltd.).

A method for applying the solution for forming a lubricating layer is not particularly limited, and examples thereof include a spin coating method, a spraying method, a paper coating method, and a dipping method.

In a case of using a dipping method, it is possible to use, for example, a method shown below. First, the substrate 11 on which the individual layers up to the protective layer 17 have been formed is immersed in the solution for forming a lubricating layer that has been placed in an immersion vessel of a dip coater. Next, the substrate 11 is lifted from the immersion vessel at a predetermined speed. As a result, the solution for forming a lubricating layer is applied to the surface of the protective layer 17 on the substrate 11.

The use of the dipping method makes it possible to uniformly apply the solution for forming a lubricating layer to the surface of the protective layer 17 and makes it possible to form the lubricating layer 18 on the protective layer 17 in a uniform film thickness.

In the present embodiment, a heat treatment is preferably carried out on the substrate 11 on which the lubricating layer 18 has been formed. The heat treatment improves the adhesion properties between the lubricating layer 18 and the protective layer 17 and improves the adhesive force between the lubricating layer 18 and the protective layer 17.

The heat treatment temperature is preferably set to 100° C. to 180° C. When the heat treatment temperature is 100° C., or higher, an effect on improvement in the adhesion properties between the lubricating layer 18 and the protective layer 17 can be sufficiently obtained. In addition, when the heat treatment temperature is set to 180° C., or lower, it is possible to prevent thermal decomposition of the lubricating layer 18. The heat treatment time is preferably set to 10 to 120 minutes.

In the present embodiment, in order to further improve the adhesive force of the lubricating layer 18 with respect to the protective layer 17, the lubricating layer 18 of the substrate 11 before or after the heat treatment may be irradiated with ultraviolet (UV) rays.

The magnetic recording medium 10 of the present embodiment includes at least the magnetic layer 16, the protective layer 17, and the lubricating layer 18 sequentially provided on the substrate 11. In the magnetic recording medium 10 of the present embodiment, the lubricating layer 18 containing the above-described fluorine-containing ether compound is formed in contact with the protective layer 17. This lubricating layer 18 cover the surface of the protective layer 17 with a high coating rate even if the lubricating layer is thin. Accordingly, in the magnetic recording medium 10 of the present embodiment, the environmental substance that generates contamination substances such as ionic impurities is prevented from intruding through gaps in the lubricating layer 18. In addition, the lubricating layer 18 in the magnetic recording medium 10 of the present embodiment has excellent wear resistance. For this reason, the magnetic recording medium 10 of the present embodiment has excellent reliability and durability.

### EXAMPLES

Hereinafter, the present invention will be described more specifically with reference to examples and comparative examples. The present invention is not limited to the following examples.

### Example 1J

A compound represented by Formula (A) above (in Formula (A), ba1 and ba2 indicating an average degree of polymerization are 4.5 and ca1 and ca2 indicating an average degree of polymerization are 4.5) was obtained through the method shown below.

First, 1,3,5-cyclohexanetriol was reacted with epibromohydrin to synthesize a compound represented by Formula (40) below. In addition, a compound represented by Formula (11) below was synthesized by protecting one hydroxyl group of 1,3-propanediol with tetrahydropyran and then reacting it with epibromohydrin.

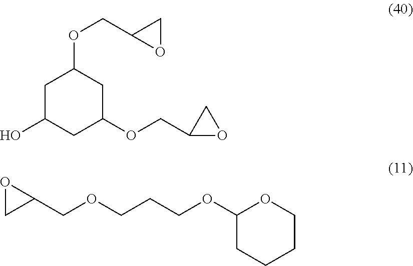 [SMILES: OC1CC(OCC2CO2)CC(OCC2CO2)C1 ; C1CCC(OCCCOCC2CO2)OC1]

Fluoropolyether represented by HOCH₂CF₂O(CF₂CF₂O)₃(CF₂O)ₜCF₂CH₂OH (in the formula, s indicating an average degree of polymerization was 4.5 and t, indicating an average degree of polymerization was 4.5) (number-average molecular weight: 1.000, molecular weight distribution: 1.1) (40.0 g), a compound represented by Formula (11) above (6.10 g), and tertiary butyl alcohol (t-BuOH) (40.0 mL) were added to a 200 mL eggplant flask under a nitrogen gas atmosphere and stirred at room temperature until the mixture became uniform. Furthermore, potassium tertiary butoxide (t-BuOK) (1.35 g) was added to the above-described eggplant flask, heated to 70° C., and stirred for 18 hours to cause a reaction.

Thereafter, the obtained reaction product was cooled to 25° C., water was added thereto. VERTREL (registered trademark) XF (hereinafter sometimes referred to as “VERTREL Xf”) manufactured by Dupont-Mitsui Fluorochemicals Co., Ltd. was further added thereto as a solvent, and an organic layer was extracted and washed with water. Anhydrous sodium sulfate was added to the organic layer for dehydration, the drying agent was filtered off, and then the filtrate was concentrated. The residue was purified through silica gel column chromatography to obtain a compound represented by Formula (12) below (17.5 g).

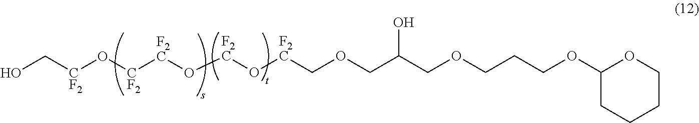

(In Formula (12), s indicating an average degree of polymerization is 4.5, and t indicating an average degree of polymerization is 4.5.)

The compound represented by Formula (12) above (17.5 g), the compound represented by Formula (40) above (2.67 g), and tertiary butyl alcohol (t-BuOH) (65.0 mL) were added to a 200 mL eggplant flask in a nitrogen gas atmosphere and stirred until the mixture became uniform at room temperature. Furthermore, potassium tertiary butoxide (t-BuOK) (0,470 g) was added to the above-described eggplant flask, heated to 70° C., and stirred for 23 hours to cause a reaction.

Thereafter, the obtained reaction product was cooled to 25° C., and water (3.3 mL) and 5% to 10% hydrochloric acid/methanol (trade name: X0041, hydrogen chloride-methanol reagent (5% to 10%) manufactured by Tokyo Chemical industry Co., Ltd.) (21.5 mL) were added thereto and stirred at room temperature for 3 hours. 5% sodium bicarbonate water (100 mL) was added to the obtained residue, extraction was performed with ethyl acetate, and the organic layer was washed with water. Thereafter, anhydrous magnesium sulfate was added to the organic layer for dehydration, the drying agent was filtered off, and then the filtrate was concentrated. The residue was purified through silica gel column chromatography to obtain 8.1 g of a compound (A).

¹H-NMR and ¹⁹F-NMR measurements of the obtained compound (A) were carried out, and the structure was identified from the following results.

¹H-NMR (acetone-d₆): δ [ppm]=1.2 to 2.0 (10H), 3.20 to 4.20 (46H)

¹⁹F-NMR (acetone-d₆): δ [ppm]=−51.99 to −55.72 (18F), −78.4 g (4F), −80.66 (4F), −89.16 to −91.14 (36F)

### Example 2

A compound represented by Formula (B) above (in Formula (B), bb1 and bb2 indicating an average degree of polymerization are 4.5 and cb1 and cb2 indicating an average degree of polymerization are 4.5) was obtained through the method shown below.

First, 1,2,3-cyclohexanetriol was reacted with epibromohydrin to synthesize a compound represented by Formula (13) below.

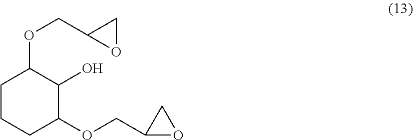 [SMILES: OC1C(OCC2CO2)CCCC1OCC1CO1]

Then, the same operation as in Example 1 was carried out except that 6.10 g of the compound represented by Formula (13) was used instead of the compound represented by Formula (40), thereby obtaining 9.5 g of a compound (B).

¹H-NMR measurement of the obtained compound (B) was carried out, and the structure was identified from the following results.

¹H-NMR (acetone-d₆): δ [ppm]=1.2 to 20 (10H), 3.20 to 4.20 (46H)

### Example 31

A compound represented by Formula (C) above (in Formula (C), bc1 and bc2 indicating an average degree of polymerization are 4.5 and cc1 and cc2 indicating an average degree of polymerization are 4.5) was obtained through the method shown below.

First, 1,2,4-cyclohexanetriol was reacted with epibromohydrin to synthesize a compound represented by Formula (14) below.

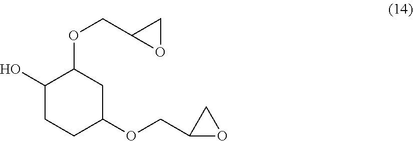 [SMILES: OC1CCC(OCC2CO2)CC1OCC1CO1]

Then, the same operation as in Example t was carried out except that 5.10 g of the compound represented by Formula (14) was used instead of the compound represented by Formula (40), thereby obtaining 10.9 g of a compound (C).

¹H-NMR measurement of the obtained compound (C) was carried out, and the structure was identified from the following results.

¹H-NMR (acetone-d₆): δ [ppm]=1.2 to 2.0 (10H), 3.20 to 4.20 (46H)

### Example 4

A compound represented by Formula (D) above (in Formula (D), bd1 and bd2 indicating an average degree of polymerization are 4.5 and cd1 and cd2 indicating an average degree of polymerization are 4.5) was obtained through the method shown below.

First, 1,2,4-cyclohexanetriol, epibromohydrin, and 2-(bromopropoxy)tetrahydro-2H-pyran were reacted with each other to synthesize a compound represented by Formula (15) below.

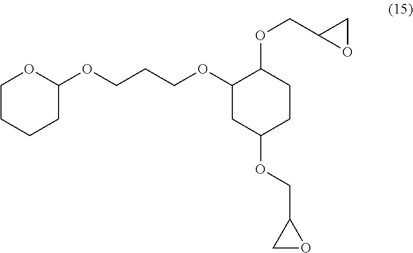 [SMILES: C1CCC(OCCCOC2CC(OCC3CO3)CCC2OCC2CO2)OC1]

Then, the same operation as in Example 1 was carried out except that 6.10 g of the compound represented by Formula (15) was used instead of the compound represented by Formula (40), thereby obtaining 10.9 g of a compound (D).

¹H-NMR measurement of the obtained compound (D) was carried out, and the structure was identified from the following results.

¹H-NMR (acetone-d₆): δ [ppm]=1.2 to 2.0 (12H), 3.20 to 4.20 (50H)

### Example 5

A compound represented by Formula (E) above (in Formula (E), be1 and be2 indicating an average degree of polymerization are 4.5 and ce1 and ce2 indicating an average degree of polymerization are 4.5) was obtained through the method shown below.

First, 1,2,4-cyclohexanetriol, epibromohydrin, and 2-(bromoethoxy)tetrahydro-2H-pyran were reacted with each other to synthesize a compound represented by Formula (16) below.

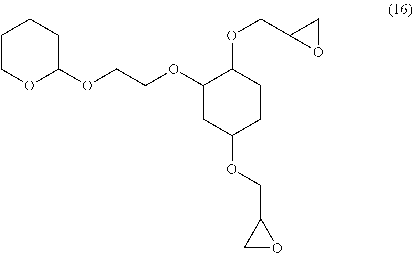 [SMILES: C1CCC(OCCOC2CC(OCC3CO3)CCC2OCC2CO2)OC1]

Then, the same operation as in Example 1 was carried out except that 6.78 g of the compound represented by Formula (16) was used instead of the compound represented by Formula (40), thereby obtaining 11.3 g of a compound (E).

¹H-NMR measurement of the obtained compound (E) was carried out, and the structure was identified from the following results.

¹H-NMR (acetone-d₆): δ [ppm]=12 to 2.0 (10H), 3.20 to 4.20 (50H)

### Example 6

A compound represented by Formula (F) above (in Formula (F), bf1 and bf2 indicating an average degree of polymerization are 4.5 and cf1 and cf2 indicating an average degree of polymerization are 4.5) was obtained through the method shown below.

First, 1,2-cyclopentanediol, epibromohydrin, and dihydropyran were reacted with each other to synthesize a compound represented by Formula (17) below.

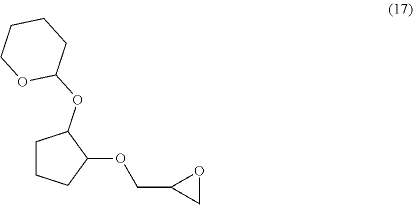 [SMILES: C1CCC(OC2CCCC2OCC2CO2)OC1]

The compound represented by Formula (12) above (17.5 g), the compound represented by Formula (17) above (3.67 g), and tertiary butyl alcohol (t-BuOH) (65.0 mL) were added to a 20 mL eggplant flask in a nitrogen gas atmosphere and stirred until the mixture became uniform at room temperature. Furthermore, potassium tertiary butoxide (t-BuOK) (0.470 g) was added to the above-described eggplant flask, heated to 70° C., and stirred for 23 hours to cause a reaction.

Thereafter, the obtained reaction product was cooled to 25° C., water was added thereto, VERTREL XF manufactured by Dupont-Mitsui Fluorochemicals Co., Ltd. was further added thereto as a solvent, and an organic layer was extracted and washed with water. Anhydrous sodium sulfate was added to the organic layer for dehydration, the drying agent was filtered off, and then the filtrate was concentrated.

This residue (16.4 g) was cooled to 0° C., N,N-dimethylfomamide (30 ml.) and sodium hydride (1.5 g) were added thereto, and the mixture was stirred at 0° C. for 1 hour. Furthermore, epibromohydrin (3 mL) was added dropwise thereto at 0° C. and stirred until the mixture became uniform, then the temperature was raised to 25° C. and the mixture was stirred for 15 hours to cause a reaction.

Thereafter, water was added to the obtained reaction product. VERTREL XF manufactured by Dupont-Mitsui Fluorochemicals Co., Ltd. was further added thereto as a solvent, and an organic layer was extracted and washed with water. Anhydrous sodium sulfate was added to the organic layer for dehydration, the drying agent was filtered off, and then the filtrate was concentrated.

The compound represented by Formula (12) above (3.67 g) and tertiary butyl alcohol (t-BuOH) (65.0 mL) were added to the residue (15.3 g) and stirred until the mixture became uniform at room temperature. Furthermore, potassium tertiary butoxide (t-BuOK) (0.270 g) was added to the above-described eggplant flask, heated to 70° C., and stirred for 23 hours to cause a reaction.

Thereafter, the obtained reaction product was cooled to 25° C., and water (3.3 ml) and 5% to 10% hydrochloric acid/methanol (20.3 mL) were added thereto and stirred at room temperature for 4 hours, 5% sodium bicarbonate water (100 mL) was added to the obtained residue, extraction was performed with ethyl acetate, and the organic layer was washed with water. Thereafter, anhydrous magnesium sulfate was added to the organic layer for dehydration, the drying agent was filtered off, and then the filtrate was concentrated. The residue was purified through silica gel column chromatography to obtain 4.1 g of a compound (F).

¹H-NMR measurement of the obtained compound (F) was carried out, and the structure was identified from the following results.

¹H-NMR (acetone-d₆): δ [ppm]=1.2 to 2.0 (10H), 3.20 to 4.20 (44H)

### Example 7

A compound represented by Formula (G) above (in Formula (G), bg1 and bg2 indicating an average degree of polymerization are 4.5 and cg1 and cg2 indicating an average degree of polymerization are 4.5) was obtained through the method shown below.

First, 1,3-cyclopentanediol, epibromohydrin, and dihydropyran were reacted with each other to synthesize a compound represented by Formula (18) below.

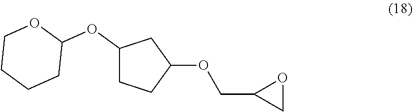 [SMILES: C1CCC(OC2CCC(OCC3CO3)C2)OC1]

Then, the same operation as in Example 6 was carried out except that 4.01 g of the compound represented by Formula (18) was used instead of the compound represented by Formula (17), thereby obtaining 5.2 g of a compound (G).

¹H-NMR measurement of the obtained compound (G) was carried out, and the structure was identified from the following results.

¹H-NMR (acetone-d₆): δ [ppm]=1.2 to 2.0 (10H), 3.20 to 4.20 (44H)

### Example 8

A compound represented by Formula (H) above (in Formula (H), bh1 and bh2 indicating an average degree of polymerization are 4.5 and ch1 and ch2 indicating an average degree of polymerization are 4.5) was obtained through the method shown below.

First, 1,3-cyclopentanediol, epibromohydrin, and 2-(bromoethoxy)tetrahydro-2H-pyran were reacted with each other to synthesize a compound represented by Formula (19) below.

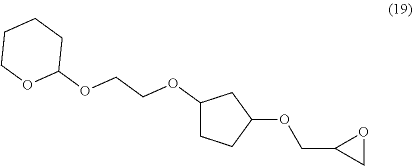 [SMILES: C1CCC(OCCOC2CCC(OCC3CO3)C2)OC1]

Then, the same operation as in Example 6 was carried out except that 2.89 g of the compound represented by Formula (19) was used instead of the compound represented by Formula (17), thereby obtaining 3.3 g of a compound (H).

¹H-NMR measurement of the obtained compound (H) was carried out, and the structure was identified from the following results.

¹H-NMR (acetone-d₆): δ [ppm]=1.2 to 2.0 (10H), 3.20 to 4.20 (48H)

### Example 9

A compound represented by Formula (I) above (in Formula (1), bi1 and bi2 indicating an average degree of polymerization are 6.5) was obtained through the method shown below.

First, 1,3,5-cyclohexanetriol, epibromohydrin, and 2-(bromopropoxy)tetrahydro-2H-pyran were reacted with each other to synthesize a compound represented by Formula (20) below. In addition, a compound represented by Formula (21) below was synthesized by protecting one hydroxyl group of 1,4-butanediol with tetrahydropyran and then reacting it with epibromohydrin.

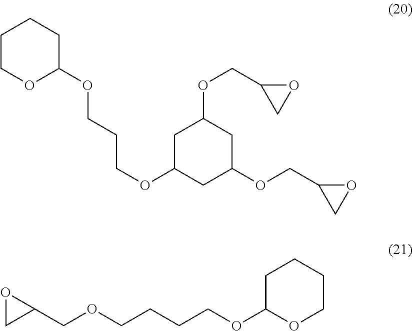 [SMILES: C1CCC(OCCCOC2CC(OCC3CO3)CC(OCC3CO3)C2)OC1 ; C(CCOC1CCCCO1)COCC1CO1]

Fluoropolyether represented by HOCH₂CF₂O(CF₂CF₂O)ₜCF₂CH₂OH (in the formula, u indicating an average degree of polymerization was 6.5) (number-average molecular weight: 1,000, molecular weight distribution: 1.1) (40.0 g), a compound represented by Formula (21) above (6.10 g), and tertiary butyl alcohol (t-BuOH) (40.0 nil..) were added to a 200 mL eggplant flask under a nitrogen gas atmosphere and stirred until the mixture became uniform at room temperature. Furthermore, potassium tertiary butoxide (t-BuOK) (1.35 g) was added to the above-described eggplant flask, heated to 70° C., and stirred for 18 hours to cause a reaction.

Thereafter, the obtained reaction product was cooled to 2.5° C., water was added thereto, VERTREL XF was further added thereto as a solvent, and an organic layer was extracted and washed with water. Anhydrous sodium sulfate was added to the organic layer for dehydration, the drying agent was filtered off, and then the filtrate was concentrated. The residue was purified through silica gel column chromatography to obtain a compound represented by Formula (22) below (14.5 g).

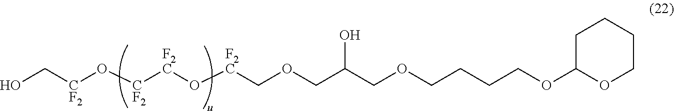

(In Formula (22), u indicating an average degree of polymerization was 6.5.)

The compound represented by Formula (22) above (14.5 g), the compound represented by Formula (20) above (3.67 g), and tertiary butyl alcohol (t-BuOH) (65.0 ml.) were added to a 200 mL eggplant flask in a nitrogen gas atmosphere and stirred until the mixture became uniform at room temperature. Furthermore, potassium tertiary butoxide (t-BuOK) (0.470 g) was added to the above-described eggplant flask, heated to 70° C., and stirred for 47 hours to cause a reaction.

Thereafter, the obtained reaction product was cooled to 25° C., and water (3.5 ml) and 5% to 10% hydrochloric acid/methanol (trade name: X0041, hydrogen chloride-methanol reagent (5% to 10%) manufactured by Tokyo Chemical Industry Co., Ltd.) (22.5 ml) were added thereto and stirred at room temperature for 3 hours. 5% sodium bicarbonate water (100 mL) was added to the obtained residue, extraction was performed with ethyl acetate, and the organic layer was washed with water. Thereafter, anhydrous magnesium sulfate was added to the organic layer for dehydration, the drying agent was filtered off, and then the filtrate was concentrated. The residue was purified through silica gel column chromatography to obtain 7.1 g of a compound (1).

¹H-NMR and ¹⁹F-NMR measurements of the obtained compound (1) were carried out, and the structure was identified from the following results.

¹H-NMR (acetone-d₆): δ [ppm]=1.2 to 2.0 (16H), 3.20 to 4.20 (50H)

¹⁹F-NMR (acetone-d₆): δ [ppm]=−77.85 to −79.00 (8F), −88.50 to −91.22 (52F)

### Example 101

A compound represented by Formula (J) above (in Formula (J), ej1 and ej2 indicating an average degree of polymerization are 4.5) was obtained through the method shown below.

First, a product obtained by reacting 3-allyloxy-1,2-propanediol-2-methoxymethyl ether with 2-(bromoethoxy)tetrahydro-2H-pyran was oxidized to synthesize a compound represented by Formula (23) below.

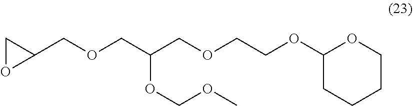 [SMILES: COCOC(COCCOC1CCCCO1)COCC1CO1]

Fluoropolyether represented by HOCH₂CF₂CF₂O(CF₂CF₂CF₂O)ₜCF₂CF₂CH₂OH (in the formula, v indicating an average degree of polymerization was 4.5) (number-average molecular weight: 1.000, molecular weight distribution: 1.1) (40.0 g), a compound represented by Formula (23) above (6.10 g), and tertiary butyl alcohol (t-BuOH) (40.0 mL) were added to a 200 mL eggplant flask under a nitrogen gas atmosphere and stirred until the mixture became uniform at room temperature. Furthermore, potassium tertiary butoxide (t-BuOK) (1.35 g) was added to the above-described eggplant flask, heated to 70° C., and stirred for 19 hours to cause a reaction.

Thereafter, the obtained reaction product was cooled to 25° C., water was added thereto. VERTREL XF was further added thereto as a solvent, and an organic layer was extracted and washed with water. Anhydrous sodium sulfate was added to the organic layer for dehydration, the drying agent was filtered off, and then the filtrate was concentrated. The residue was purified through silica gel column chromatography to obtain a compound represented by Formula (24) below (13.5 g).

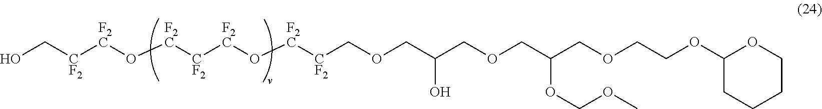

(In Formula (24), v indicating an average degree of polymerization was 4.5.)

The compound represented by Formula (24) above (13.5 g), the compound represented by Formula (40) above (3.37 g), and tertiary butyl alcohol (t-BuOH) (65.0 mL) were added to a 200 mL, eggplant flask in a nitrogen gas atmosphere and stirred until the mixture became uniform at room temperature. Furthermore, potassium tertiary butoxide (t-BuOK) (0.570 g) was added to the above-described eggplant flask, heated to 70° C., and stirred for 25 hours to cause a reaction.

Thereafter, the obtained reaction product was cooled to 25° C., and water (3.5 mL) and 5% to 10% hydrochloric acid/methanol (trade name: X0041, hydrogen chloride-methanol reagent (5% to 10%) manufactured by Tokyo Chemical Industry Co., Ltd.) (22.5 mL) were added thereto and stirred at room temperature for 3 hours. 5% sodium bicarbonate water (100 mL) was added to the obtained residue, extraction was performed with ethyl acetate, and the organic layer was washed with water. Thereafter, anhydrous magnesium sulfate was added to the organic layer for dehydration, the drying agent was filtered off, and then the filtrate was concentrated. The residue was purified through silica gel column chromatography to obtain 6.5 g of a compound (J).

¹H-NMR and ¹⁹F-NMR measurements of the obtained compound (J) were carried out, and the structure was identified from the following results.

¹H-NMR (acetone-d₆): δ [ppm]=1.2 to 2.0 (6H), 3.20 to 4.20 (58H)

¹⁹F-NMR (acetone-d₆): δ [ppm]=−82.66 to −84.00 (40F), −85.16 to −86.91 (8F), −123.16 to −124.91 (8F), −128.47 to −130.20 (8F)

### Example 11

A compound represented by Formula (K) above (in Formula (K), bk1 and bk2 indicating an average degree of polymerization are 4.5 and ck1 and ck2 indicating an average degree of polymerization are 45) was obtained through the method shown below.

The compound represented by Formula (12) above (17.5 g), the compound represented by Formula (17) above (3.67 g), and tertiary butyl alcohol (t-BuOH) (65.0 mL) were added to a 200 mL eggplant flask in a nitrogen gas atmosphere and stirred until the mixture became uniform at room temperature. Furthermore, potassium tertiary butoxide (t-BuOK) (0.470 g) was added to the above-described eggplant ask, heated to 70° C., and stirred for 23 hours to cause a reaction.

Thereafter, the obtained reaction product was cooled to 25° C., water was added thereto. VERTREL XF manufactured by Dupont-Mitsui Fluorochemicals Co., Ltd. was further added thereto as a solvent, and an organic layer was extracted and washed with water. Anhydrous sodium sulfate was added to the organic layer for dehydration, the drying agent was filtered off, and then the filtrate was concentrated.

Dichloromethane (200 ml.) and 4-dimethylaminopyridine (0.0 g) were added to this residue (16.4 g) and cooled to 0° C. Furthermore, triethylamine (10 mL) was added dropwise thereto and stirred at 0° C. until the mixture became uniform, and then methanesulfonyl chloride (3.0 mL) was added dropwise thereto, the temperature was raised to 25° C., and the mixture was stirred for 6 hours to cause a reaction.

Thereafter, water was added to the obtained reaction product. VERTREL XF manufactured by Dupont-Mitsui Fluorochemicals Co., Ltd. was further added thereto as a solvent, and an organic layer was extracted and washed with water. Anhydrous sodium sulfate was added to the organic layer for dehydration, the drying agent was filtered off, and then the filtrate was concentrated.

The compound represented by Formula (12) above (3.67 g) and tertiary butyl alcohol (t-BuOH) (65.0 mL) were added to the residue (15.3 g) and stirred until the mixture became uniform at room temperature. Furthermore, potassium tertiary butoxide (t-BuOK) (0.470 g) was added to the above-described eggplant flask, heated to 70° C., and stirred for 23 hours to cause a reaction.

Thereafter, the obtained reaction product was cooled to 25° C., and water (3.3 mL) and 5% to 10% hydrochloric acid/methanol (20.3 mL) were added thereto and stirred at room temperature for 4 hours. 5% sodium bicarbonate water (100 nit.) was added to the obtained residue, extraction was performed with ethyl acetate, and the organic layer was washed with water. Thereafter, anhydrous magnesium sulfate was added to the organic layer for dehydration, the drying agent was filtered off, and then the filtrate was concentrated. The residue was purified through silica gel column chromatography to obtain 4.1 g of a compound (K).

¹H-NMR measurement of the obtained compound (K) was carried out, and the structure was identified from the following results.

¹H-NMR (acetone-d₆): δ [ppm]=1.2 to 2.0 (10H), 3.20 to 4.20 (38H)

### Example 12

A compound represented by Formula (L) above (in Formula (L), bl1 and bl2 indicating an average degree of polymerization are 4.5 and cl1 and cl2 indicating an average degree of polymerization are 4.5) was obtained through the method shown below.

First, allyl bromide and allyl alcohol were reacted with a product obtained by reacting 1,3-cyclopentanediol, dihydropyran, and epibromohydrin with each other, and oxidized to synthesize a compound represented by Formula (25) below.

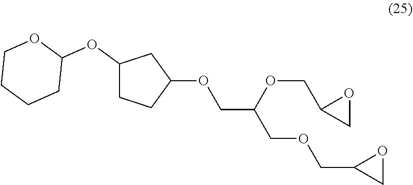 [SMILES: C1CCC(OC2CCC(OCC(COCC3CO3)OCC3CO3)C2)OC1]

Then, the same operation as in Example 1 was carried out except that 2.64 g of the compound represented by Formula (25) was used instead of the compound represented by Formula (40), thereby obtaining 10.9 g of a compound (L).

¹H-NMR measurement of the obtained compound (L) was carried out, and the structure was identified from the following results.

¹H-NMR (acetone-d₆): δ [ppm]=1.2 to 2.0 (10H), 3.20 to 4.20 (50H)

### Example 13

A compound represented by Formula (M) above (in Formula (M), bm1 and bm2 indicating an average degree of polymerization are 4.5 and cm1 and cm2 indicating an average degree of polymerization are 4.5) was obtained through the method shown below.

First, a product obtained by reacting 3-allyloxy-1,2-propanediol-2-methoxymethyl ether with 2-(bromopropoxy)tetrahydro-2H-pyran was oxidized to synthesize a compound represented by Formula (26) below.

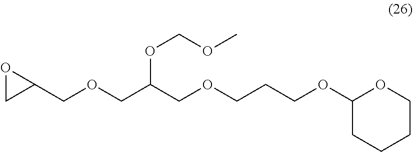 [SMILES: COCOC(COCCCOC1CCCCO1)COCC1CO1]

Then, the same operation as when synthesizing the compound represented by Formula (12) in Example 1 was carried out except that 6.70 g of the compound represented by Formula (26) was used instead of the compound represented by Formula (11), thereby obtaining a compound represented by Formula (27) below.

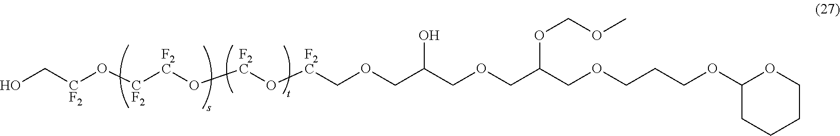

(In Formula (27), s indicating an average degree of polymerization is 4.5, and t indicating an average degree of polymerization is 4.5.)

Then, the same operation as in Example 1 was carried out except that 15.2 g of the compound represented by Formula (27) was used instead of the compound represented by Formula (12), thereby obtaining 7.3 g of a compound (M).

¹H-NMR measurement of the obtained compound (M) was carried out, and the structure was identified from the following results.

¹H-NMR (acetone-d₆): 1.2 to 2.0 (10H), 3.20 to 4.20 (58H)

### Example 14

A compound represented by Formula (N) above (in Formula (N), bn1 and bn2 indicating an average degree of polymerization are 4.5 and cn1 and cn2 indicating an average degree of polymerization are 4.5) was obtained through the method shown below.

First, a product obtained by protecting 3-buten-1-ol with a tetrahydropyranyl (THP) group was oxidized to synthesize a compound represented by Formula (28) below.

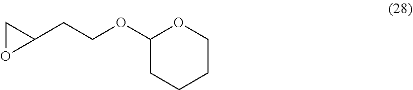 [SMILES: C1CCC(OCCC2CO2)OC1]

Then, the same operation as when synthesizing the compound represented by Formula (12) in Example 1 was carried out except that 6.30 g of the compound represented by Formula (28) was used instead of the compound represented by Formula (11), thereby obtaining a compound represented by Formula (29) below.

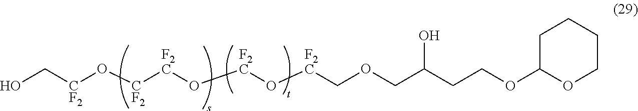

(In Formula (29), s indicating an average degree of polymerization is 4.5, and t indicating an average degree of polymerization is 4.5.)

Then, the same operation as in Example 1 was carried out except that 14.8 g of the compound represented by Formula (29) was used instead of the compound represented by Formula (12), thereby obtaining 7.0 g of a compound (N).

¹H-NMR measurement of the obtained compound (N) was carried out, and the structure was identified from the following results.

¹H-NMR acetone-d₆): 1.2 to 2.0 (10H), 3.20 to 4.20 (38H)

### Example 15

A compound represented by Formula (O) above tin Formula (O), bo1 and bo2 indicating an average degree of polymerization are 4.5 and co1 and co2 indicating an average degree of polymerization are 4.5) was obtained through the method shown below.

First, epibromohydrin was reacted with a product obtained by protecting one hydroxyl group of 2,2-difluoro-1,3-propanediol with a THP group to synthesize a compound represented by Formula (30) below.

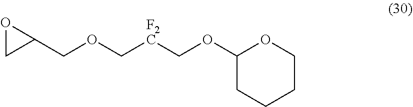 [SMILES: FC(F)(COCC1CO1)COC1CCCCO1]

Then, the same operation as when synthesizing the compound represented by Formula (12) in Example 1 was carried out except that 6.4 g of the compound represented by Formula (30) was used instead of the compound represented by Formula (11), thereby obtaining a compound represented by Formula (31) below.

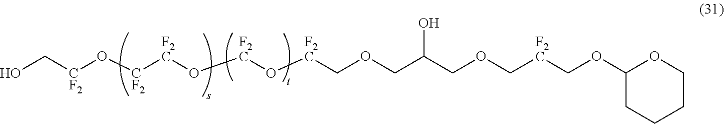

(In Formula (31), s indicating an average degree of polymerization is 4.5, and t indicating an average degree of polymerization is 4.5.)

Then, the same operation as in Example 1 was carried out except that 15.6 g of the compound represented by Formula (31) was used instead of the compound represented by Formula (12), thereby obtaining 7.5 g of a compound (0).

¹H-NMR and ¹⁹F-NMR measurements of the obtained compound (0) were carried out, and the structure was identified from the following results.

¹H-NMR (acetone-d₆): 1.2 to 2.0 (6H), 3.20 to 4.20 (46H)

¹⁹F-NMR (acetone-d₆): δ [ppm]=−51.99 to −55.72 (9F), −78.48 (2F), −80.66 (2F), −89.16 to −91.14 (18F), −144.28 (4F)

### Example 16

A compound represented by Formula (P) above (in Formula (P), bp1 and bp2 indicating an average degree of polymerization are 4.5 and cp1 and cp2 indicating an average degree of polymerization are 4.5) was obtained through the method shown below.

First, allyl bromide and allyl alcohol were reacted with a product obtained by reacting 3-amino-cyclopentane-1-ol with epibromohydrin, and oxidized to synthesize a compound represented by Formula (32) below.

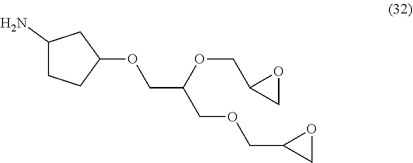 [SMILES: NC1CCC(OCC(COCC2CO2)OCC2CO2)C1]

Then, the same operation as in Example 1 was carried out except that 2.89 g of the compound represented by Formula (32) was used instead of the compound represented by Formula (40), thereby obtaining 11.1 g of a compound (P).

¹H-NMR measurement of the obtained compound (P) was carried out, and the structure was identified from the following results.

¹H-NMR (acetone-d₆): δ [ppm]=1.2 to 2.0 (10H), 3.20 to 4.20 (51H)

### Comparative Example 1

A compound represented by Formula (Q) below was obtained through the method shown below.

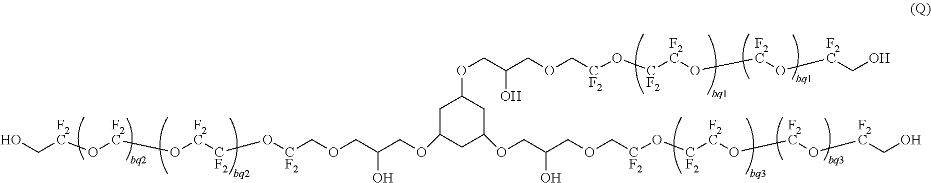

(In Formula (Q), bq1, bq2, and bq3 indicating an average degree of polymerization are 4.5, and cq1, cq2, and cq3 indicating an average degree of polymerization are 4.5.)

First, 1,3,5-cyclohexanetriol was reacted with epibromohydrin to synthesize a compound represented by Formula (33) below.

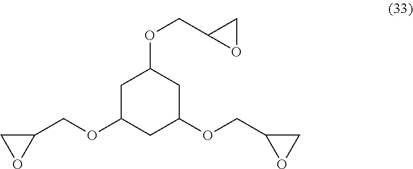 [SMILES: C1OC1COC1CC(OCC2CO2)CC(OCC2CO2)C1]

Fluoropolyether represented by HOCH₂CF₂CF₂O(CF₂CF₂O)ₜCF₂CH₂OH (in the formula, s indicating an average degree of polymerization was 4.5 and t indicating an average degree of polymerization was 4.5) (number-average molecular weight: 1,000, molecular weight distribution: 1.1) (40.0 g), a compound represented by Formula (33) above (3.10 g), and tertiary butyl alcohol (t-BuOH) (40.0 mL) were added to a 200 mL eggplant flask under a nitrogen gas atmosphere and stirred until the mixture became uniform at room temperature. Furthermore, potassium tertiary butoxide (t-BuOK) (2.55 g) was added to the above-described eggplant flask, heated to 70° C., and stirred for 50 hours to cause a reaction.

Thereafter, the obtained reaction product was cooled to 25° C., water was added thereto, VERTREL XF was further added thereto as a solvent, and an organic layer was extracted and washed with water. Anhydrous sodium sulfate was added to the organic layer for dehydration, the drying agent was filtered off, and then the filtrate was concentrated. The residue was purified through silica gel column chromatography to obtain a compound represented by Formula (Q) above (15.5 g).

¹H-NMR and ¹⁹F-NMR measurements of the obtained compound (Q) were carried out, and the structure was identified front the following results.

¹H-NMR (acetone-d₆): δ [ppm]=1.2 to 2.0 (6H), 3.20 to 4.20 (33H)

¹⁹F-NMR (acetone-d₆): δ [ppm]=−90.57 to −88.88 (36F), −83.21 to −81.20 (6F), −80.64 to −78.64 (6F), −53.32 to −51.94 (18F)

### Comparative Example 2

A compound represented by Formula (R) below was obtained through the method shown below.

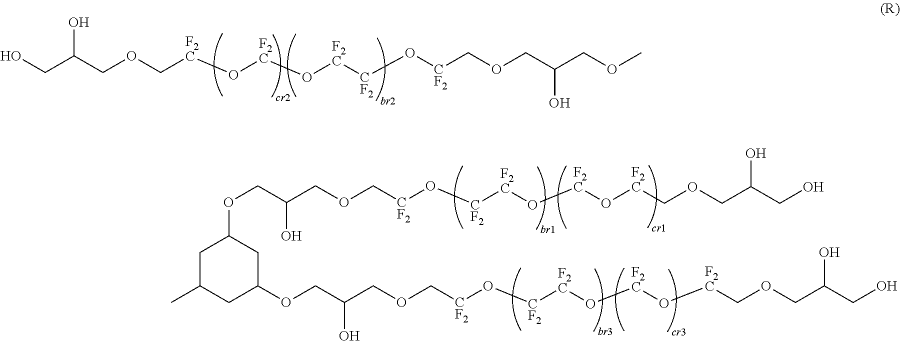

(In Formula (R), br1, br2, and br3 indicating an average degree of polymerization are 4.5, and cr1, cr2, and cr3 indicating an average degree of polymerization are 4.5.)

36 mL of t-butanol and 4 g of the compound (Q) above were added to a 50 mL eggplant flask in a nitrogen gas atmosphere and stirred until these became uniform to obtain a mixture. Next, 0.4 g of potassium tert-butoxide was added to the above-described mixture, and 300 μL. of glycidol was added thereto while heating to 70° C. and stirred for 5 hours to cause a reaction.

Thereafter, the reaction solution after the reaction was cooled to 25° C. and neutralized with hydrochloric acid, and then a fluorine solvent (trade name: ASAHIKLIN AK-225 manufactured by Asahi Glass Co., Ltd.) was added thereto, and the mixture was washed with water. Anhydrous sodium sulfate was added to an organic layer of the reaction solution after washing with water for dehydration, the drying agent was filtered off, and then the filtrate was concentrated. The residue was purified through silica gel column chromatography to obtain a compound represented by Formula (R) above (2.0 g).

¹H-NMR and ¹⁹F-NMR measurements of the obtained compound (R) were carried out, and the structure was identified from the following results.

¹H-NMR (acetone-d₆): δ [ppm]=1.211 (3H), 2.523 (3H), 3.452 (3H), 3.574 (3H), 3.625 (3H), 3.744 (6H), 3.797 to 3.901 (12H), 3.927 to 4.116 (18H)

¹⁹F-NMR (acetone-d₆): δ [ppm]=−90.87 to −89.19 (36F), −81.43 to −78.90 (12F), −55.63 to −52.29 (18F)

### Comparative Example 31

A compound represented by Formula (S) below was obtained through the method shown below.

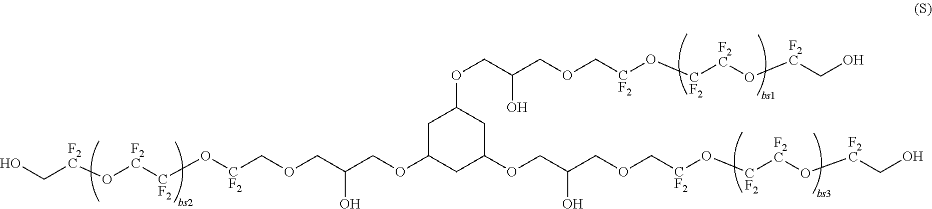

(In Formula (S), bs1, bs2, and bs3 indicating an average degree of polymerization were 6.5.)

The same operation as in Comparative Example 1 was carried out except that 40.0 g of fluoropolyether represented by HOCH₂CF₂O(CF₂CF₂O)ₜCF₂CH₂OH (in the formula, u indicating an average degree of polymerization was 6.5) (number-average molecular weight: 1,000, molecular weight distribution: 1.1) was used instead of fluoropolyether represented by HOCH₂CH₂CF₂O(CF₂CF₂O)ₛ(CF₂)ₜCF₂CH₂OH (in the formula, s indicating an average degree of polymerization was 4.5 and t indicating an average degree of polymerization was 4.5) (number-average molecular weight: 1,000, molecular weight distribution: 1.1) used in Comparative Example 1, thereby obtaining 11.1 g of a compound (S).

¹H-NMR and ¹⁹F-NMR measurements of the obtained compound (S) were carried out, and the structure was identified from the following results.

¹H-NMR (acetone-d₆): δ [ppm]=1.185 (3H), 2.499 (3H), 3.427 (3H), 3.554 (3H), 3.600 (3H), 3.709 (6H), 3.735 to 3.918 (12H), 4.029 to 4.078 (18H)

¹⁹F-NMR (acetone-D₆): δ [ppm]=89.07 (60F), −81.37 (6F), −78.85 (6F)

### Comparative Example 4

A compound represented by Formula (f) below was obtained through the method shown below.

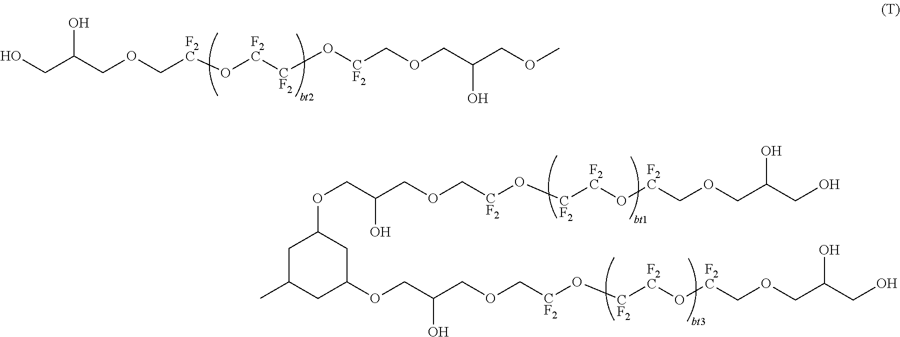

(In Formula (T), bt1, bt2, and bt3 indicating an average degree of polymerization were 6.5.)

The same operation as in Comparative Example 2 was carried out except that 4.0 g of the compound represented by Formula (S) was used instead of the compound represented by Formula (Q) used in Comparative Example 2, thereby obtaining 1.8 g of a compound (T).

¹H-NMR and ¹⁹F-NMR measurements of the obtained compound (T) were carried out, and the structure was identified from the following results.

¹H-NMR (acetone-d₆): δ [ppm]=1.211 (3H), 2.523 (3H), 3.452 (314), 3.574 (3H), 3.625 (31), 3.744 (614), 3.797 to 3.901 (12H), 3.927 to 4.116 (18H)

¹⁹F-NMR (acetone-D₆): δ [ppm]=−88.67 (60F), −78.43 (12F)

### Comparative Example 5

A compound represented by Formula (U) below was obtained through the method shown below,

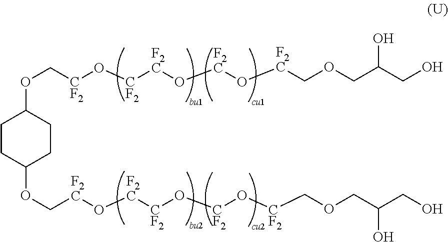

(In Formula (U), bu1 and bu2 indicating an average degree of polymerizations are 4.5, and cu1 and cu2 indicating an average degree of polymerization are 4.5.)

First, glycidol was protected with a tetrahydropyranyl (THP) group to synthesize a compound represented by Formula (34) below.

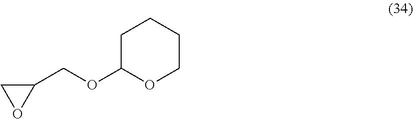 [SMILES: C1CCC(OCC2CO2)OC1]

Fluoropolyether represented by HOCH₂CF₂O(CF₂CF₂O)ₛ(CF₂O)ₜCF₂CH₂OH (in the formula, s indicating an average degree of polymerization was 4.5 and t indicating an average degree of polymerization was 4.5) (number-average molecular weight: 1,000, molecular weight distribution: 1.1) (40.0 g), a compound represented by Formula (34) above (2.10 g), and tertiary butyl alcohol (t-BuOH) (40.0 mL) were added to a 200 mL eggplant flask under a nitrogen gas atmosphere and stirred until the mixture became uniform at room temperature. Furthermore, potassium tertiary butoxide (t-BuOK) (1.55 g) was added to the above-described eggplant flask, heated to 70° C., and stirred for 50 hours to cause a reaction.

Thereafter, the obtained reaction product was cooled to 25° C., water was added thereto, VERTREL XF was further added thereto as a solvent, and an organic layer was extracted and washed with water. Anhydrous sodium sulfate was added to the organic layer for dehydration, the drying agent was filtered off, and then the filtrate was concentrated. The residue was purified through silica gel column chromatography to obtain a compound represented by Formula (35) below (20.3 g).

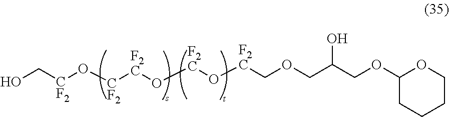

(In Formula (35), s indicating an average degree of polymerization is 4.5, and t indicating an average degree of polymerization is 4.5.)

N,N-dimethylformamide (200 mL) and the compound represented by Formula (35) (20.3 g) were mixed, cooled to 0° C., and stirred, and then sodium hydride (1.0 g) was added thereto. After the mixture was further stirred at 0° C. for 2 hours, 1,4-dichlorocyclohexane (5.0 g) was added thereto, the temperature was raised to 25° C. and the mixture was stirred for 6 hours to cause a reaction.

Thereafter, water (3.3 mL) and 5% to 10% hydrochloric acid/methanol (20.3 mL) were added to the obtained reaction product and stirred at room temperature for 2 hours. 5% sodium bicarbonate water (100 mL) was added to the obtained residue, extraction was performed with ethyl acetate, and the organic layer was washed with water. Thereafter, anhydrous magnesium sulfate was added to the organic layer for dehydration, the drying agent was filtered off, and then the filtrate was concentrated. The residue was purified through silica gel column chromatography to obtain 10.2 g of a compound (U).

¹H-NMR, and ¹⁹F-NMR measurement of the obtained compound (U) were carried out, and the structure was identified from the following results.

¹H-NMR (acetone-d₆): δ [ppm]=1.2 to 2.0 (8H), 3.20 to 4.20 (24H)

¹⁹F-NMR (acetone-d₆): δ [ppm]=−90.87 to −89.19 (24F), −81.43 to −78.90 (8F), −55.63 to −52.29 (12F)

### Comparative Example 6

A compound represented by Formula (V) below was produced through the method shown below.

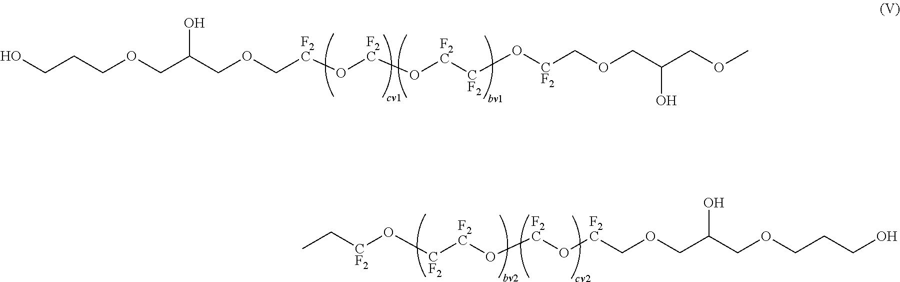

(In Formula (V), bv1 and bv2 indicating an average degree of polymerization are 4.5, and cv1 and cv2 indicating an average degree of polymerization are 4.5.)

The same operation as in Example 1 was carried out except that 4.20 g of epibromohydrin was used instead of the compound represented by Formula (40), thereby obtaining 8.5 g of a compound (V).

¹H-NMR and ¹⁹F-NMR measurements of the obtained compound (V) were carried out, and the structure was identified front the following results.

¹H-NMR (acetone-d₆): δ [ppm]=1.2 to 2.0 (10H), 3.20 to 4.20 (46H)

¹⁹F-NMR (acetone-d₆): δ [ppm]=−55.6 to −50.6 (18F), −77.7 (4F), −80.3 (4F), −91.0 to −88.5 (36F)

The structures of R¹ and R⁵, the structures of R² and R⁴, and the structure of R³ (X or X′ which is an alicyclic structure, Y, and a substituent in X or X′ in Formulae (2-1) to (2-4)) when the compounds of Examples 1 to 16 thus obtained are adapted to Formula (1) are shown in Table 1.

TABLE 1

| | R [symbol] | | | | | | |
|---|---|---|---|---|---|---|---|
| | | | Substiment | | | | |
| | X or X [symbol] | Y | in X or X [symbol] | Formula | R¹ and R⁴ | R¹ and R⁵ | Compound |
| Example 1 | Cyclohexane | —O— | —OH | (2-1) | Formula (4) | Formula (10) | A |
| | | | | | b = 4.5 | j = 1 | |
| | | | | | c = 4.5 | k = 1 | |
| Example 2 | Cyclohexane | —O— | —OH | (2-1) | Formula (4) | Formula (10) | B |
| | | | | | b = 4.5 | j = 1 | |
| | | | | | c = 4.5 | k = 1 | |
| Example 3. | Cyclohexane | —O— | —OH | (2-1) | Formula (4) | Formula (10) | C |
| | | | | | b = 4.5 | j = 1 | |
| | | | | | c = 4.5 | k = 1 | |
| Example 4 | Cyclohexane | —O— | —OC₃H₆OH | (2-1) | Formula (4) | Formula (10) | D |
| | | | | | b = 4.5 | j = 1 | |
| | | | | | c = 4.5 | k = 1 | |
| Example 5 | Cyclohexane | —O— | —OC₂H₄OH | (2-1) | Formula (4) | Formula (10) | E |
| | | | | | b = 4.5 | j = 1 | |
| | | | | | c = 4.5 | k = 1 | |
| Example 6 | Cyclopentane | —O— | —OH | (2-3) | Formula (4) | Formula (10) | F |
| | | | | | b = 4.5 | j = 1 | |
| | | | | | c = 4.5 | k = 1 | |
| Example 7 | Cyclopentane | —O— | —OH | (2-3) | Formula (4) | Formula (10) | G |
| | | | | | b = 4.5 | j = 1 | |
| | | | | | c = 4.5 | k = 1 | |
| Example 8 | Cyclopentane | —O— | —OC₂H₄OH | (2-3) | Formula (4) | Formula (10) | H |
| | | | | | b = 4.5 | j = 1 | |
| | | | | | c = 4.5 | k = 1 | |
| Example 9 | Cyclohexane | —O— | —OC₃H₆OH | (2-1) | Formula (4) | Formula (10) | I |
| | | | | | b = 6.5 | j = 1 | |
| | | | | | c = 0 | k = 2 | |
| Example 10 | Cyclohexane | —O— | —OH | (2-1) | Formula (6) | Formula (10) | J |
| | | | | | [symbol] = 4.5 | f = 2 | |
| | | | | | | g = 1 | |
| Example 11 | Cyclopentane | —O— | —OH | (2-2) | Formula (4) | Formula (10) | K |
| | | | | | b = 4.5 | j = 1 | |
| | | | | | c = 4.5 | k = 1 | |
| Example 12 | Cyclopentane | —O— | —OH | (2-4) | Formula (4) | Formula (10) | L |
| | | | | | b = 4.5 | j = 1 | |
| | | | | | c = 4.5 | k = 1 | |
| Example 13 | Cyclohexane | —O— | —OH | (2-1) | Formula (4) | Formula (10) | M |
| | | | | | b = 4.5 | j = 2 | |
| | | | | | c = 4.5 | k = 1 | |
| Example 14 | Cyclohexane | —O— | —OH | (2-1) | Formula (4) | Formula (9) | N |
| | | | | | b = 4.5 | i = 1 | |
| | | | | | c = 4.5 | | |
| Example 15 | Cyclohexane | —O— | —OH | (2-1) | Formula (4) | Formula (8) | O |
| | | | | | b = 4.5 | h = 1 | |
| | | | | | c = 4.5 | | |
| Example 16 | Cyclopentane | —O— | —NH₂ | (2-4) | Formula (4) | Formula (10) | P |
| | | | | | b = 4.5 | j = 1 | |
| | | | | | c = 4.5 | k = 1 | |

[symbol]

indicates data missing or illegible when filed

The number-average molecular weights (Mn) of the compounds of Examples 1 to 16 and Comparative Examples 1 to 6 were obtained by the above-described ¹H-NMR and/or ¹⁹F-NMR measurements. The results are shown in Table 2.

It is inferred that, in the values of the average molecular weight of the synthesized compounds, variations of approximately 1 to 5 may exist depending on, for example, the molecular weight distributions of the fluoropolyether used as a raw material of the compound, and differences in the operation at the time of synthesizing the compounds.

TABLE 2

| | | Number | | | | |
|---|---|---|---|---|---|---|
| | | average | Film | Si | Wear | |
| | | molecular | thickness | adsorption | resistance | Comprehensive |
| | Compound | weight | (Å) | amount | test | evaluation |
| Example 1 | (A) | 2,541 | 8.0 | 0.47 | ◯ | ◯ |
| Example 2 | (B) | 2,548 | 8.5 | 0.45 | ◯ | ◯ |
| Example 3 | (C) | 2,539 | 9.0 | 0.47 | ◯ | ◯ |
| Example 4 | (D) | 2,611 | 9.0 | 0.44 | ⊙ | ◯ |
| Example 5 | (E) | 2,576 | 8.0 | 0.44 | ⊙ | ◯ |
| Example 6 | (F) | 2,508 | 8.5 | 0.46 | ◯ | ◯ |
| Example 7 | (G) | 2,523 | 8.0 | 0.45 | ◯ | ◯ |
| Example 8 | (H) | 2,426 | 8.5 | 0.41 | ⊙ | ◯ |
| Example 9 | (I) | 2,610 | 9.0 | 0.51 | ⊙ | ◯ |
| Example 10 | (J) | 2,466 | 9.0 | 0.33 | ⊙ | ◯ |
| Example 11 | (K) | 2,311 | 9.0 | 0.51 | ◯ | ◯ |
| Example 12 | (L) | 2,466 | 8.5 | 0.38 | ◯ | ◯ |
| Example 13 | (M) | 2,600 | 8.5 | 0.34 | ◯ | ◯ |
| Example 14 | (N) | 2,501 | 8.0 | 0.44 | ◯ | ◯ |
| Example 15 | (O) | 2,587 | 8.5 | 0.52 | ⊙ | ◯ |
| Example 16 | (P) | 2,456 | 8.5 | 0.39 | ◯ | ◯ |
| Comparative | (Q) | 3,330 | 8.5 | 1.00 | ◯ | X |
| Example 1 | | | | | | |
| Comparative | (R) | 3,400 | 8.5 | 0.95 | Δ | X |
| Example 2 | | | | | | |
| Comparative | (S) | 3,332 | 9.0 | 1.02 | ◯ | X |
| Example 3 | | | | | | |
| Comparative | (T) | 3,412 | 8.5 | 0.92 | Δ | X |
| Example 4 | | | | | | |
| Comparative | (U) | 2,258 | 8.5 | 0.87 | X | X |
| Example 5 | | | | | | |
| Comparative | (V) | 2,328 | 9.0 | 0.85 | Δ | X |
| Example 6 | | | | | | |

Next, solutions for forming a lubricating layer were prepared using the compounds obtained in Examples 1 to 16 and Comparative Examples 1 to 6 by the method shown below. Moreover, lubricating layers of magnetic recording media were formed using the obtained solutions for forming a lubricating layer by the method hown below, and magnetic recording media of Examples 1 to 16 and Comparative Examples 1 to 6 were obtained.

“Solutions for Forming Lubricating Layer”

The compounds obtained in Examples 1 to 16 and Comparative Examples 1 to 6 were each dissolved in VERTREL (registered trademark) XF (trade name, manufactured by Dupont-Mitsui Fluorochemicals Co., Ltd.), which is a fluorine-based solvent, diluted with VERTREL such that the film thicknesses became 8 Å to 9 Å when applied onto protective layers, and used as solutions for forming a lubricating layer.

“Magnetic Recording Media”

Magnetic recording media each having an adhesive layer, a soft magnetic layer, a first underlayer, a second underlayer, a magnetic layer, and a protective layer sequentially provided on a substrate having a diameter of 65 mm were prepared. As the protective layer, one made of carbon was used.

The solutions for forming a lubricating layer of Examples 1 to 16 and Comparative Examples 1 to 6 were each applied onto the protective layers of the magnetic recording media in which the individual layers up to the protective layer had been formed by the dipping method. The dipping method was carried out under conditions of an immersion speed of 10 mm/sec, an immersion time of 30 seconds and a lifting speed of 1.2 mm/sec.

Thereafter, the magnetic recording media coated with the solutions for forming a lubricating layer were each placed in a thermostatic vessel set at 120° C., heated for 10 minutes, and solvents in the solutions for forming a lubricating layer were removed to form lubricating layers on the protective layers and obtain magnetic recording media.

The film thicknesses of the lubricating layers in the magnetic recording media of Examples 1 to 16 and Comparative Examples 1 to 6 thus obtained were measured using FT-IR (trade name: Nicolet iS50, manufactured by Thermo Fisher Scientific Inc.). The results are shown in Table 2.

(Chemical Substance Resistance Test)

Contamination of the magnetic recording media due to environmental substances that generate contamination substances in a high-temperature environment was investigated through an evaluation method shown below. In the evaluation method shown below. Si ions were used as an environmental substance to measure the Si adsorption amount as the amount of a contamination substance which is generated by the environmental substance and contaminates the magnetic recording media.

Specifically, the magnetic recording media to be evaluated were held for 240 hours in the presence of siloxane-based Si rubber in a high-temperature environment at 85° C., and a humidity of 0%. Next, the adsorption amount of Si present on the surfaces of the magnetic recording media was analyzed and measured through secondary ion mass spectrometry (SIMS), and the degree of contamination due to Si ions was evaluated as the Si adsorption amount. The Si adsorption amount was evaluated with a numerical value when the result of Comparative Example 1 was set to 1.00. The results are shown in Table 2.

Next, the magnetic recording media of Examples 1 to 16 and Comparative Examples 1 to 6 were subjected to a wear resistance test shown below.

(Wear Resistance Test)

Using a pin-on-disk type friction and wear tester, an alumina ball with a diameter of 2 mm as a contactor was slid on a lubricating layer of each of the magnetic recording media at a load of 40 gf and a sliding speed of 0.25 n/sec, and the friction coefficient of the surface of the lubricating layer was measured. Then, the sliding time until the friction coefficient of the surface of the lubricating layer increased suddenly was measured. The sliding time until the friction coefficient increased suddenly was measured 4 times for the lubricating layer of each of the magnetic recording media, and an average value (time) thereof was used as an index of the wear resistance of the lubricant coating film. The results of the magnetic recording media of Examples 1 to 16 and Comparative Examples 1 to 6 are shown in Table 2. The time until the friction coefficient increased suddenly was evaluated as follows. The larger the numerical value, the better the result.
⊙ (Excellent): 880 sec or longer
◯ (Favorable). 780 sec or longer and shorter than 880 sec
Δ (Acceptable): 680 see or longer and shorter than 780 see
x (Poor): shorter than 680 sec

The time until the friction coefficient increased suddenly can be used as an index of the wear resistance of the lubricating layers due to reasons shown below. This is because the lubricating layers of the magnetic recording media wear as the magnetic recording media are used, and when the lubricating layers are lost due to wear, the contactors and the protective layers come into direct contact with each other, resulting in a sharp increase in the friction coefficient. It is thought that the time until this friction coefficient increases suddenly has a correlation with the friction test.

(Comprehensive Evaluation)

Based on the results of the chemical substance resistance test and the wear resistance test, a comprehensive evaluation was performed according to the following criteria.
◯ (Favorable): The Si adsorption amount in the chemical substance resistance test was 0.60 or less, and the evaluation of the wear resistance test was ⊙ (excellent) or ◯ (favorable),
x (Poor): The criteria for the above ◯ (favorable) were not satisfied.

As shown in Table 2, the magnetic recording media of Examples 1 to 16 had a lower Si adsorption amount and better resistance to chemical substances than the magnetic recording media of Comparative Examples 1 to 6. In addition, the magnetic recording media of Examples 1 to 16 had a long sliding time until the friction coefficient increased sharply, and had a favorable wear resistance. Thus, the comprehensive evaluation of all of Examples 1 to 16 was ◯ (favorable).

In particular, in Examples 10 and 13 in which there are three hydroxyl groups in each of R¹ and R⁵, the Si adsorption amount was 035 or less, resulting in favorable resistance to chemical substances. It is inferred that this is due to the following reasons. The polar groups in R¹, R³, and R⁵ have a pinning effect that prevents the bulky alicyclic structure contained in R¹ from being lifted completely from the protective layer. In Examples 10 and 13, since there are three hydroxyl groups in each of R¹ and R⁵, the pinning effect can be more effectively obtained. As a result, it is inferred that an appropriate distance between the lubricating layer and the protective layer was maintained, resulting in favorable resistance to chemical substances.

In addition, in all of Examples 4, 5, 8, and 9 in which the alicyclic structure contained in R¹ has a substituent consisting of an alkoxy group having a hydroxyl group at a terminal, the wear resistance was favorable. It is inferred that this is because the distance between the alicyclic structure X in R³ and carbon atom to which the hydroxyl group in the substituent is bound is sufficiently ensured by a linking group which contains an ether bond and a carbon atom and has moderate flexibility, whereby the pinning effect of the alicyclic structure X due to the hydroxyl group in the substituent is appropriate.

In addition, in Example 10 in which R² and R⁴ are Formula (6) and Example 15 in which R¹ and R⁵ are Formula (8), the wear resistance was also favorable.

On the other hand, the comprehensive evaluation of all of Comparative Examples 1 to 6 was x (poor).

More specifically, in all of Comparative Example 1 in which the compound (Q) having three perfluoropolyether chains in the molecule was used. Comparative Example 2 in which the compound (R) was used. Comparative Example 3 in which the compound (S) was used, and Comparative Example 4 in which the compound (T) was used, the result of the chemical substance resistance test was inferior compared to Examples 1 to 16.

In addition, in Comparative Example 2 in which the compound (R) in which each of the terminal groups corresponding to R¹ and R⁵ in Formula (1) contained two hydroxyl groups, individual hydroxyl groups are bound to different carbon atoms, and the carbon atoms to which the hydroxyl groups are bound are bound to each other was used, the result of the wear resistance test was inferior compared to Comparative Example 1 in which the compound (Q) in which each of the terminal groups is one hydroxyl group was used.

Furthermore, in Comparative Example 4 in which the compound (T) in which each of the terminal groups corresponding to R¹ and R⁵ in Formula (1) contained two hydroxyl groups, individual hydroxyl groups are bound to different carbon atoms, and the carbon atoms to which the hydroxyl groups are bound are bound to each other was used, the result of the wear resistance test was inferior compared to Comparative Example 3 in which the compound (S) in which each of the terminal groups is one hydroxyl group was used.

In Comparative Example 5 in which the compound (U) in which the organic group corresponding to R³ in Formula (1) did not contain a polar group was used, the evaluation of the chemical substance resistance test was inferior and the result of the wear resistance test was also inferior compared to Examples 1 to 16.

In addition, in Comparative Example 6 in which the compound (V) in which the organic group corresponding to R³ in Formula (1) did not contain an alicyclic structure having 3 to 13 carbons was used, the result of the chemical substance resistance test was inferior and the result of the wear resistance test was also inferior compared to Examples 1 to 16.

Based on the above results, it was found that by forming lubricating layers containing the compounds of Examples 1 to 16 on the protective layers of the magnetic recording media, the lubricating layers obtained had excellent resistance to chemical substances and wear resistance even if the thickness of the lubricating layers was as thin as 8 Å to 9 Å.

### INDUSTRIAL APPLICABILITY

The present invention provides a fluorine-containing ether compound with which a lubricating layer having excellent resistance to chemical substances and wear resistance can be formed even if the lubricating layer is thin.

By using a lubricant for a magnetic recording medium containing the fluorine-containing ether compound of the present invention, it is possible to form a lubricating layer having excellent resistance to chemical substances and wear resistance even if the lubricating layer is thin.

### REFERENCE SIGNS LIST

10 Magnetic recording media
11 Substrate
12 Adhesive layer
13 Soft magnetic layer
14 First underlayer
15 Second underlayer
16 Magnetic layer
17 Protective layer
18 Lubricating layer

1. A fluorine-containing ether compound represented by Formula (1) below,
R¹—CH₂—R²—CH₂—R³—CH₂—R⁴—CH₂—R⁵ (1)
(in Formula (1), R³ is a divalent organic group containing at least one polar group and an alicyclic structure having 3 to 13 carbons, and does not contain a perfluoropolyether chain, R² and R⁴ are perfluoropolyether chains, and R¹ and R⁵ are terminal groups containing two or three polar groups, in which individual polar groups are bound to different carbon atoms and the carbon atoms to which the polar groups are bound are bound to each other via a linking group containing a carbon atom to which the polar groups are not bound).

2. The fluorine-containing ether compound according to claim 1,
wherein R³ above is represented by any of Formulae (2-1) to (2-4) below, 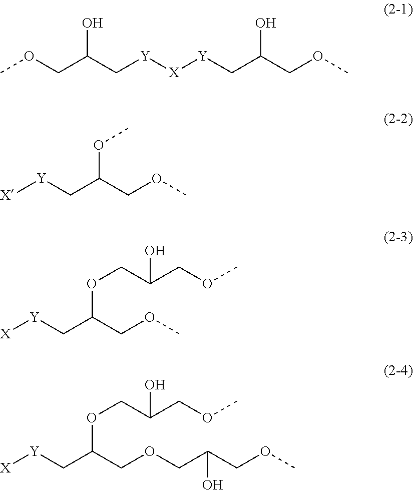
(in Formula (2-1), X is an alicyclic structure having 3 to 13 carbons, and Y represents —O—, —NH—, or —CH₂—),
(in Formula (2-2), X′ is an alicyclic structure having 3 to 13 carbons and has at least one substituent containing a polar group, and Y represents —O—, —NH—, or —CH₂—),
(in Formula (2-3), X is an alicyclic structure having 3 to 13 carbons, and Y represents —O—, —NH—, or —CH₂—),
(in Formula (2-4), X is an alicyclic structure having 3 to 13 carbons, and Y represents —O—, —NH—, or —CH₂—),

3. The fluorine-containing ether compound according to claim 2,
wherein Y in Formulae (2-1) to (2-4) is —O—.

4. The fluorine-containing ether compound according to claim 1,
wherein the alicyclic structure contained in R³ above is a saturated alicyclic structure.

5. The fluorine-containing ether compound according to claim 1,
wherein the alicyclic structure contained in R³ above is selected from a group consisting of cyclopropane, cyclobutane, cyclopentane, cyclohexane, cycloheptane, cyclooctane, cyclononane, cyclodecane, cycloundecane, cyclododecane, cyclotridecane, and adamantane.

6. The fluorine-containing ether compound according to claim 1,
wherein the at least one polar group contained in R³ above is a group containing a polar group selected from a group consisting of a hydroxyl group, an alkoxy group, an amide group, an amino group, a carbonyl group, a carboxy group, a nitro group, a cyano group, and a sulfo group.

7. The fluorine-containing ether compound according to claim 1,
wherein R² and R⁴ above are any of Formulae (4) to (6) below,
—CF₂O—(CF₂CF₂O)_{b}—(CF₂O)_{c}—CF₂— (4)
(b and c in Formula (4) indicate an average degree of polymerization, each independently representing 0 to 30, provided that b and c are not 0 at the same time),
—CF(CF₃)—(OCF(CF₃)CF₂)_{d}—OCF(CF₃)— (5)
(d in Formula (5) indicates an average degree of polymerization and represents 0.1 to 30), and
—CF₂CF₂O—(CF₂CF₂CF₂O)ₑ—CF₂CF₂— (6)
(e in Formula (6) indicates an average degree of polymerization and represents 0.1 to 30).

8. The fluorine-containing ether compound according to claim 1,
wherein the two or three polar groups contained in R¹ and R⁵ above are all hydroxyl groups.

9. The fluorine-containing ether compound according to claim 1,
wherein R¹ and R⁵ above are terminal groups represented by any of Formulae (7) to (10) below, 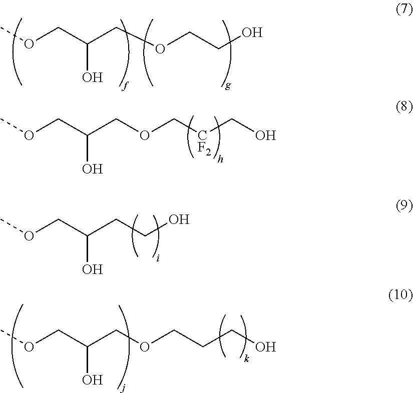
(in Formula (7), f represents an integer of 1 to 2, and g represents an integer of 1 to 5),
(in Formula (8), h represents an integer of 1 to 5),
(in Formula (9), i represents an integer of 1 to 5), and
(in Formula (10), j represents an integer of 1 to 2, and k represents an integer of 1 to 2).

10. The fluorine-containing ether compound according to claim 1,
wherein a number-average molecular weight thereof is within a range of 500 to 10,000.

11. The fluorine-containing ether compound according to claim 1,
wherein the compound represented by Formula (1) above is any of compounds represented by Formulae (A) to (P) below, 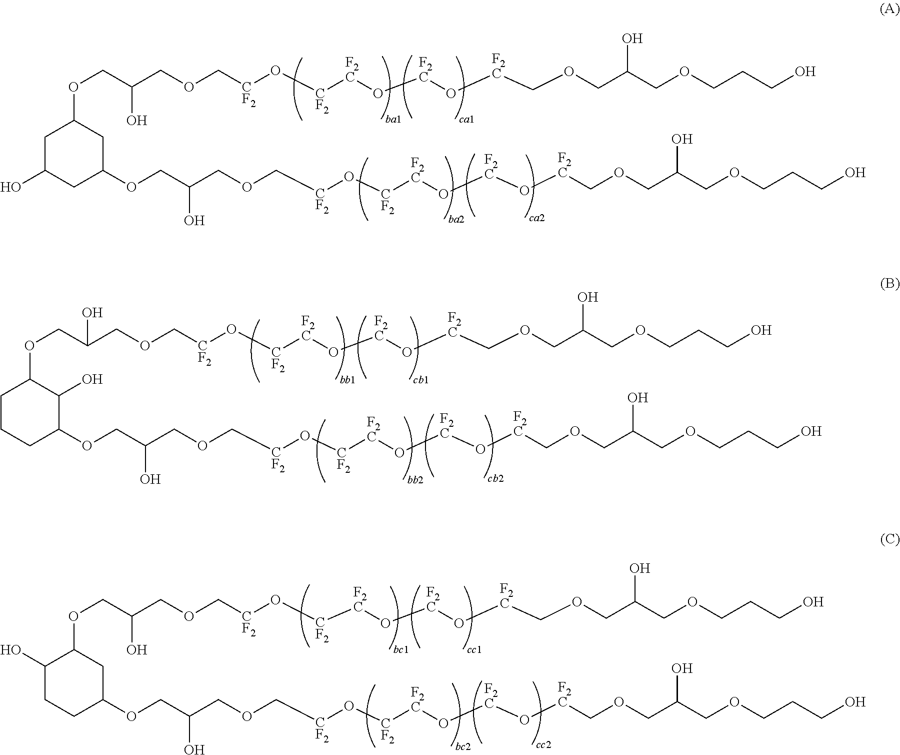
(in Formula (A), ba1, ca1, ba2, and ca2 indicate an average degree of polymerization, ba1 and ba2 represent 0 to 30, and ca1 and ca2 represent 0 to 30, provided that ba1 and ca1 are not 0 at the same time and ba2 and ca2 are not 0 at the same time),
(in Formula (B), bb1, cb1, bb2, and cb2 indicate an average degree of polymerization, bb1 and bb2 represent 0 to 30, and cb1 and cb2 represent 0 to 30, provided that bb1 and cb1 are not 0 at the same time and bb2 and cb2 are not 0 at the same time),
(in Formula (C), bc1, cc1, bc2, and cc2 indicate an average degree of polymerization, bc1 and bc2 represent 0 to 30, and cc1 and cc2 represent 0 to 30, provided that bc1 and cc1 are not 0 at the same time and bc2 and cc2 are not 0 at the same time), 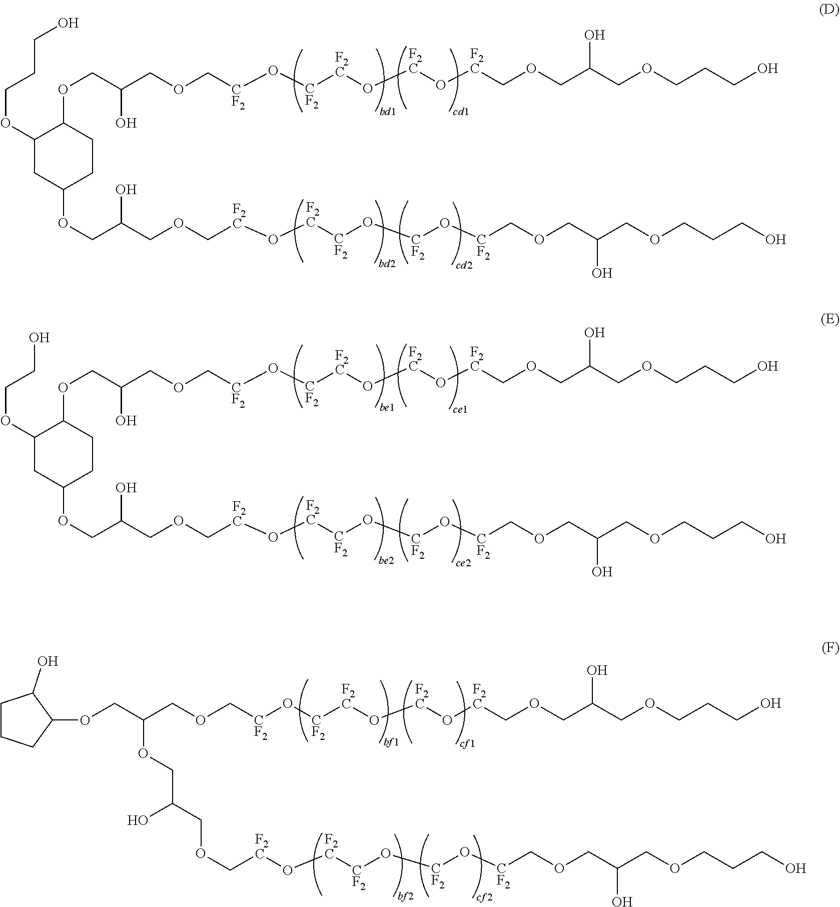
(in Formula (D), bd1, cd1, bd2, and cd2 indicate an average degree of polymerization, bd1 and bd2 represent 0 to 30, and cd1 and cd2 represent 0 to 30, provided that bd1 and cd1 are not 0 at the same time and bd2 and cd2 are not 0 at the same time),
(in Formula (E), be1, ce1, be2, and ce2 indicate an average degree of polymerization, be1 and be2 represent 0 to 30, and ce1 and ce2 represent 0 to 30, provided that be1 and ce1 are not 0 at the same time and be2 and ce2 are not 0 at the same time),
(in Formula (F), bf1, cf1, bf2, and cf2 indicate an average degree of polymerization, bf1 and bf2 represent 0 to 30, and cf1 and cf2 represent 0 to 30, provided that bf1 and cf1 are not 0 at the same time and bf2 and cf2 are not 0 at the same time), 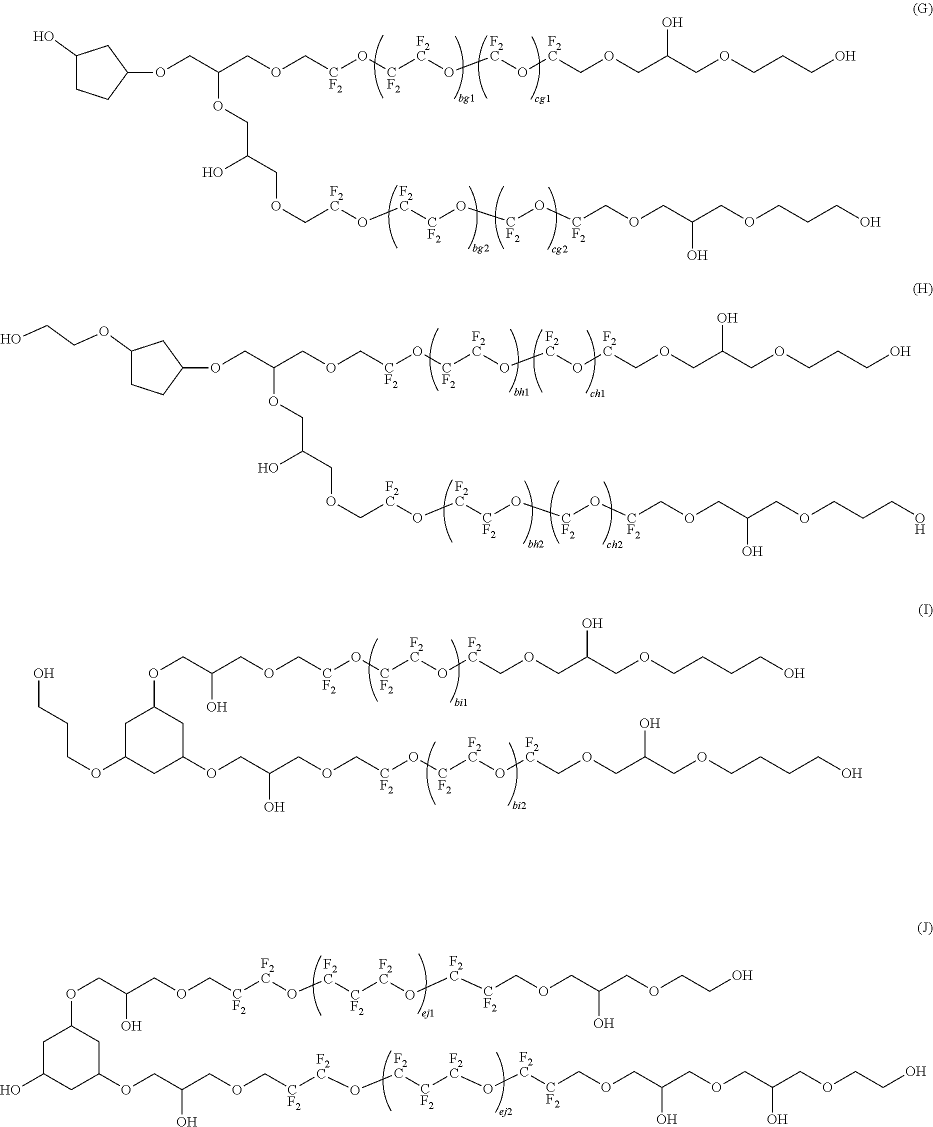
(in Formula (G), bg1, cg1, bg2, and cg2 indicate an average degree of polymerization, bg1 and bg2 represent 0 to 30, and cg1 and cg2 represent 0 to 30, provided that bg1 and cg1 are not 0 at the same time and bg2 and cg2 are not 0 at the same time),
(in Formula (H), bh1, ch1, bh2, and ch2 indicate an average degree of polymerization, bh1 and bh2 represent 0 to 30, and ch1 and ch2 represent 0 to 30, provided that bh1 and ch1 are not 0 at the same time and bh2 and ch2 are not 0 at the same time),
(in Formula (I), bi1 and bi2 indicate an average degree of polymerization and represent 0.1 to 30),
(in Formula (J), ej1 and ej2 indicate an average degree of polymerization and represent 0.1 to 30), 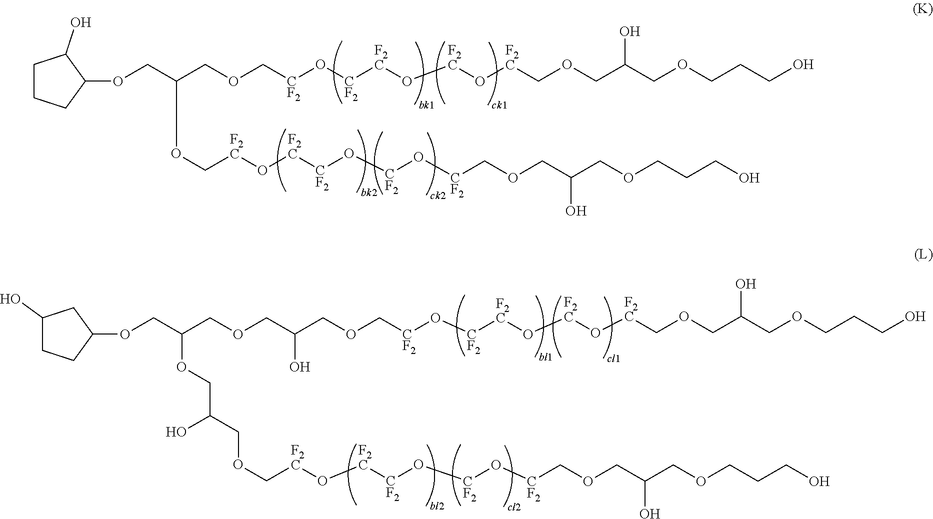
(in Formula (K), bk1, ck1, bk2, and ck2 indicate an average degree of polymerization, bk1 and bk2 represent 0 to 30, and ck1 and ck2 represent 0 to 30, provided that bk1 and ck1 are not 0 at the same time and bk2 and ck2 are not 0 at the same time),
(in Formula (L), bl1, cl1, bl2, and cl2 indicate an average degree of polymerization, bl1 and bl2 represent 0 to 30, and cl1 and cl2 represent 0 to 30, provided that bl1 and cl1 are not 0 at the same time and bl2 and cl2 are not 0 at the same time), 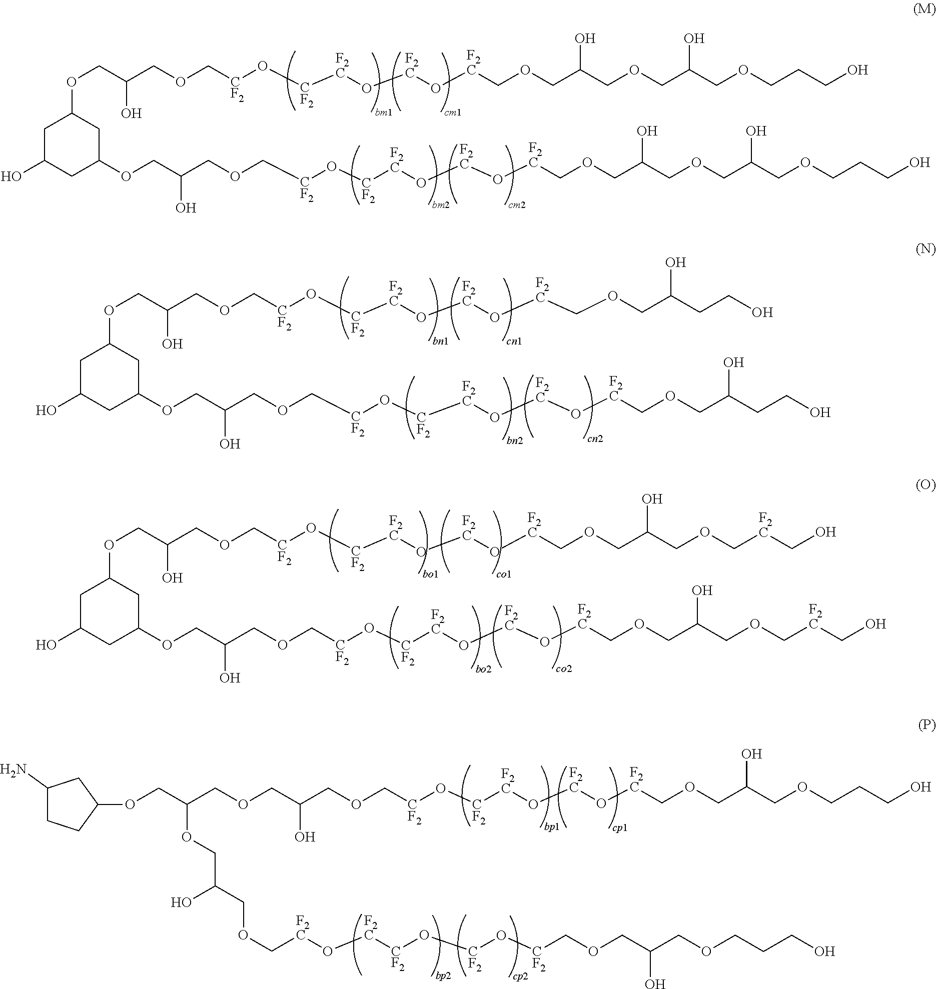
(in Formula (M), bm1, cm1, bm2, and cm2 indicate an average degree of polymerization, bm1 and bm2 represent 0 to 30, and cm1 and cm2 represent 0 to 30, provided that bm1 and cm1 are not 0 at the same time and bm2 and cm2 are not 0 at the same time),
(in Formula (N), bn1, cn1, bn2, and cn2 indicate an average degree of polymerization, bn1 and bn2 represent 0 to 30, and cn1 and cn2 represent 0 to 30, provided that bn1 and cn1 are not 0 at the same time and bn2 and cn2 are not 0 at the same time),
(in Formula (O), bo1, co1, bo2, and co2 indicate an average degree of polymerization, bo1 and bo2 represent 0 to 30, and co1 and co2 represent 0 to 30, provided that bo1 and co1 are not 0 at the same time and bo2 and co2 are not 0 at the same time), and
(in Formula (P), bp1, cp1, bp2, and cp2 indicate an average degree of polymerization, bp1 and bp2 represent 0 to 30, and cp1 and cp2 represent 0 to 30, provided that bp1 and cp1 are not 0 at the same time and bp2 and cp2 are not 0 at the same time).

12. A lubricant for a magnetic recording medium, comprising:
the fluorine-containing ether compound according to claim 1.

13. A magnetic recording medium,
wherein at least a magnetic layer, a protective layer, and a lubricating layer are sequentially provided on a substrate, and
wherein the lubricating layer contains the fluorine-containing ether compound according to claim 1.

14. The magnetic recording medium according to claim 13,
wherein an average film thickness of the lubricating layer is 0.5 nm to 2.0 nm.
